(19) United States
(12) Patent Application Publication
(10) Pub. No.: US 2023/0220480 A1
(43) Pub. Date: Jul. 13, 2023

(54) IRON-SCORE AND IN VITRO METHOD FOR IDENTIFYING HIGH RISK DLBCL SUBJECTS AND THERAPEUTIC USES AND METHODS

(71) Applicants: CENTRE NATIONAL DE LA RECHERCHE SCIENTIFIQUE (CNRS), Paris (FR); CENTRE HOSPITALIER UNIVERSITAIRE DE MONTPELLIER, Montpellier (FR); INSERM (INSTITUT NATIONAL DE LA SANTÉ ET DE LA RECHERCHE MÉDICALE), Paris (FR); UNIVERSITE DE MONTPELLIER, Montpellier (FR); INSTITUT CURIE, Paris (FR)

(72) Inventors: Jérôme MOREAUX, MONTPELLIER (FR); Raphaël RODRIGUEZ, PARIS (FR); Julie DEVIN, MONTPELLIER (FR); Caroline BRET, MONTPELLIER (FR); Tatiana CANEQUE COBO, PARIS (FR)

(73) Assignees: CENTRE NATIONAL DE LA RECHERCHE SCIENTIFIQUE (CNRS), Paris (FR); CENTRE HOSPITALIER UNIVERSITAIRE DE MONTPELLIER, Montpellier (FR); INSTITUT NATIONAL DE LA SANTÉ ET DE LA RECHERCHE MÉDICALE (INSERM), Paris (FR); UNIVERSITE DE MONTPELLIER, Montpellier (FR); INSTITUT CURIE, Paris (FR)

(21) Appl. No.: 17/774,592

(22) PCT Filed: Nov. 6, 2020
(86) PCT No.: PCT/EP2020/081349

§ 371 Date: May 5, 2022

(30) Foreign Application Priority Data
Nov. 6, 2019 (EP) 19306436.7

(51) Int. Cl. C12Q 1/6886; A61K 31/35; A61K 45/06; A61P 35/00

(52) U.S. Cl. CPC C12Q 1/6886; A61K 31/35; A61K 45/06; A61P 35/00; C12Q 2600/118; C12Q 2600/158; C12Q 2600/106

(57) ABSTRACT

The invention relates to the use of an iron-score based on the expression level of at least 2 genes, in particular at least 5, preferably at least 10, and even preferably 11 genes selected in the group consisting of ALAS1, HIF1A, LRP2, HMOX1, HMOX2, HFE, ISCA1, SLC25A37, PPOX, STEAP1 and TMPRSS6 involved in the iron metabolism, as a prognosis marker in subjects having DLBCL, in particular for identifying subjects with a poor outcome such as a relapse and/or death.

### FIELD OF THE INVENTION

The present invention relates to the field of in vitro method for prognosing the outcome of a subject affected by a B-Cell Lymphoma, in particular DLBCL, as well as associated therapeutic uses and methods.

### BACKGROUND OF THE INVENTION

Lymphomas can affect any organ in the body, present with a wide range of symptoms. They are traditionally divided into Hodgkin's lymphoma (which accounts for about 10% of all lymphomas) and non-Hodgkin lymphoma. Non-Hodgkin lymphoma represents a wide spectrum of illnesses that vary from the most indolent to the most aggressive malignancies. They arise from lymphocytes that are at various stages of development, and the characteristics of the specific lymphoma subtype reflect those of the cell from which they originated. The human mature B cell malignancies represent a medical challenge that is only partly met by current therapy, justifying concerted investigation into their molecular circuitry and pathogenesis. Each lymphoma subtype bears a phenotypic resemblance to B cells at a particular stage of differentiation, as judged by the presence or absence of immunoglobulin (Ig) variable (V) region mutations and by gene expression profiling.

The most common B-Cell lymphomas are non-Hodgkin lymphoma, in particular Diffuse large B-cell lymphoma (DLBCL), Follicular lymphoma, Marginal zone B-cell lymphoma (MZL) or Mucosa-Associated Lymphatic Tissue lymphoma (MALT), Small lymphocytic lymphoma (also known as chronic lymphocytic leukemia, CLL), and Mantle cell lymphoma (MCL).

The present invention will focus as examples to DLBCL.

Diffuse large B-cell lymphoma (DLBCL) is the most common lymphoid malignancy in adults, accounting for up to 35% of non-Hodgkin lymphomas. DLBCL is characterized by its clinical and biological heterogeneity.

Heterogeneity is reflected in transcriptionally defined subtypes that classify two main molecular groups based on the Cell-Of-Origin classification and associated with different clinical outcome. The germinal center cell-DLBCL subtype (GCB) derive from centroblasts of dark-zone of the lymph node and is associated with a better outcome whereas the activated B cell-DLBCL subtype (ABC) that derive from plasmablast cells is associated with a poor outcome (Rosenwald et al., 2002). Although DLBCL is curable with Rituximab (R)-based chemotherapy regimens, such as CHOP (cyclophosphamide, doxorubicin, vincristine and prednisone) in over 60% of patients, the remainder develop recurrent or progressive disease that is often fatal. Therefore, new therapeutic approaches are still needed to achieve an effective treatment for high risk/refractory DLBCL.

The inventors developed an Iron score in particular for DLBCL subjects, which is a gene expression profile (GEP)-based risk score based on 11 prognostic genes. Iron plays a central role in a large number of essential cellular functions, including oxygen sensing, energy metabolism, respiration and folate metabolism, and is also required for cell proliferation, serving as a cofactor for several enzymes involved in DNA synthesis and DNA repair. The iron score of the present invention allows to identify DLBCL patients with a poor outcome and that could benefit from targeted therapy. In addition, the inventors demonstrated that Ironomycin, an iron chelator, significantly reduces the median number of viable primary DLBCL cells of patients without major toxicity for non-tumor cells from the microenvironment and presented a low toxicity on hematopoietic progenitors compared to conventional treatment. Interestingly, the inventors also identified a significant synergistic effect when Ironomycin is combined with Doxorubicin, but also with Venetoclax, Idelalisib, Ibrutinib, and entospletinib.

Altogether, these data demonstrated that a subgroup of high-risk DLBCL patients could be identified with the iron score and could benefit from a treatment comprising an inhibitor of iron metabolism.

### SUMMARY OF THE INVENTION

A first object of the present invention is the use of an iron-score based on the expression level of at least 1 gene and/or protein involved in the iron metabolism, in particular at least 1 gene and/or protein encoded by the 62 genes listed in table 3, preferably at least 1 gene, in particular at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, as a prognosis marker in subjects having B-cell Lymphoma in particular DLBCL, in particular for identifying subjects with a poor outcome such as a relapse and/or death.

In a particular embodiment, the present invention concerns the use of an iron-score based on the expression level of at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, as a prognosis marker in DLBCL subjects, in particular for identifying DLBCL subjects with a poor outcome such as a relapse and/or death.

The invention also concerns an in vitro method for identifying B-cell lymphoma subject with a poor outcome, in particular DLBCL subject with a poor outcome that may benefit from a therapeutic treatment targeting iron metabolism, comprising the steps of:
a) Measuring the expression level of at least 1, in particular at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject;
b) Calculating a score value from said expression level obtained at step a),
c) Classifying and identifying the said subject as having a poor outcome according to the score value in comparison to a predetermined reference value.

In a particular embodiment, the present invention concerns an in vitro method for identifying DLBCL subjects with a poor outcome that may benefit of a therapeutic treatment targeting iron metabolism, comprising the steps of:
a) Measuring the expression level of at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject;
b) Calculating a score value from said expression level obtained at step a)
c) Classifying and identifying the said subject with a poor outcome according to the score value in comparison to a predetermined reference value.

Another subject-matter of the present invention is an in vitro method for monitoring the efficacy of a therapeutic treatment targeting iron metabolism in a subject having B-cell Lymphoma, in particular a high-risk Diffuse Large B-Cell Lymphoma (DLBCL) and undergoing said treatment, comprising the steps of:
a) Measuring the expression level of at least 1, in particular at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T1 before or during or after the subject has been administered said therapeutic treatment targeting iron metabolism;
b) Calculating a score value at time T1 from said expression level obtained at step a),
c) Measuring the expression level of at least at least 1, in particular at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T2 before or during or after the subject has been administered the said therapeutic treatment targeting iron metabolism, wherein said time T2 is posterior to said time T1;
d) Calculating a score value at time T2 from said expression level obtained at step c),
e) Assessing the efficacy of a therapeutic treatment based on the comparison the score value at T2 obtained at step d) with the score value at T1 obtained at step b).

In a particular embodiment, the invention concerns an in vitro method for monitoring the efficacy of a therapeutic treatment targeting iron metabolism in a subject having a high-risk Diffuse Large B-Cell Lymphoma (DLBCL) and undergoing said treatment, comprising the steps of:
a) Measuring the expression level of at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T1 before or during or after the subject has been administered said therapeutic treatment targeting iron metabolism;
b) Calculating a score value at time T1 from said expression level obtained at step a),
c) Measuring the expression level of at least at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T2 before or during or after the subject has been administered the said therapeutic treatment targeting iron metabolism, wherein said time T2 is posterior to said time T1;
d) Calculating a score value at time T2 from said expression level obtained at step c),
e) Assessing the efficacy of a therapeutic treatment based on the comparison the score value at T2 obtained at step d) with the score value at T1 obtained at step b).

The in vitro methods of the present invention optionally comprise one or more housekeeping gene(s) for normalization of the data.

By “housekeeping genes”, it is meant genes that are constitutively expressed at a relatively constant level across many or all known conditions, because they code for proteins that are constantly required by the cell, hence, they are essential to a cell and always present under any conditions. It is assumed that their expression is unaffected by experimental conditions.

The proteins they code are generally involved in the basic functions necessary for the sustenance or maintenance of the cell. Non-limitating examples of housekeeping genes that may be used in methods of the invention include:
HPRT1 (hypoxanthine phosphoribosyltransferase 1),
UBC (ubiquitin C),
YWHAZ (tyrosine 3-monooxygenase/tryptophan 5-monooxygenase activation protein, zeta polypeptide),
B2M (beta-2-microglobulin),
GAPDH (glyceraldehyde-3-phosphate dehydrogenase),
FPGS (folylpolyglutamate synthase),
DECR1 (2,4-dienoyl CoA reductase 1, mitochondrial),
PPIB (peptidylprolyl isomerase B (cyclophilin B)),
ACTB (actin β),
PSMB2 (proteasome (prosome, macropain) subunit, beta type, 2),
GPS1 (G protein pathway suppressor 1),
CANX (calnexin),
NACA (nascent polypeptide-associated complex alpha subunit),
TAX1BP1 (Tax1 (human T-cell leukemia virus type I) binding protein 1), and
PSMD2 (proteasome (prosome, macropain) 26S subunit, non-ATPase, 2).

When such housekeeping genes are added to the expression profile (it is not always necessary), they are used for normalization purpose. In this case, the number of housekeeping genes used for normalization in methods according to the invention is preferably comprised between one and five with a preference for three.

The in vitro methods of the present invention comprise a step of measuring the expression level of at least 1, 2, 3, 4, 5, 6, 7, 8, 9, 10, or 11 genes useful for the outcome prognostic, also named ‘prognosis genes’ or genes of interest’ according to the invention.

The present invention also relates to a kit dedicated to in vitro methods according to the invention, in particular for DLBCL subjects, comprising or consisting of reagents for determining the expression level of at least 1, preferably at least 5, more preferably at least 10 and even preferably at least 11 genes and/or proteins selected in the group consisting of HMOX2, PPOX, TMPRSS6, HFE, SLC25A37, STEAP1, ISCA1, HMOX1, LRP2, HIF1A, and ALAS1 in a sample of said subject.

The invention also relates to a pharmaceutical composition comprising, in a pharmaceutical acceptable vehicle, an molecule targeting iron metabolism in particular iron chelators and small molecules sequestering lysosomal iron, in particular selected in the group consisting of Deferasirox, Deferoxamine, Deferiprone, Salinomycin, analogs or derivatives thereof, preferably salinomycin and nitrogen-containing analogs of salinomycin, for use in a method for treating subjects having B-cell lymphoma, in particular Diffuse large B-cell lymphoma (DLBCL), Follicular lymphoma, Marginal zone B-cell lymphoma (MZL) or Mucosa-Associated Lymphatic Tissue lymphoma (MALT), Small lymphocytic lymphoma (also known as chronic lymphocytic leukemia, CLL), and Mantle cell lymphoma (MCL), and preferably subjects having DLBCL.

In a particular embodiment, the pharmaceutical composition is used in a method for treating B-Cell lymphoma subjects identified according to the in vitro method of the invention as having a poor outcome according to iron-score and consequently likely to display a B-cell Lymphoma relapse and/or death, in particular a DLBCL relapse and/or death.

Another subject-matter of the invention is a pharmaceutical product comprising:
(i) a molecule targeting iron metabolism, in particular an iron chelator or small molecule sequestering lysosomal iron and
(ii) another anti-cancer agents selected from the group consisting of agents used in chemotherapy, targeted treatments, immune therapies, and combinations thereof, as combination product for simultaneous, separate or staggered use as a medicament in the treatment of B-Cell lymphoma, preferably DLBCL, in particular in DLBCL subjects with a poor outcome according to in vitro method of the invention.

The present invention also relates to systems (and computer readable medium for causing computer systems) to perform the in vitro methods of the invention, based on above described expression levels of genes and/or proteins as identified above.

In particular, the system includes a machine-readable memory, such as a computer or/and a calculator, and a processor configured to compute R Maxstat function and Cox multivariate function, according to the invention. This system is dedicated to perform the in vitro methods according to the invention in particular for identifying B-Cell lymphoma subjects with a poor outcome.

In particular, the system 1 for analyzing a biological sample of a subject affected by a B-Cell Lymphoma, in particular DLBCL, comprises:
(a) a determination module 2 configured to receive a biological sample and to determine expression level information concerning the prognosis genes as disclosed in the present invention and optionally one or more housekeeping gene(s);
(b) a storage device 3 configured to store the expression level information from the determination module;
(c) a comparison module 4 adapted to compare the expression level information stored on the storage device with reference data, and to provide a comparison result, wherein the comparison result is indicative of the outcome of the subject; and
(d) a display module 5 for displaying a content based in part on the comparison result for the user, wherein the content is a signal indicative of the outcome of the subject.

### Definitions

The term ‘subject’ or ‘patient’ or ‘individual’ refers to a human subject, whatever its age or sex. The subject is affected by a B-Cell Lymphoma. The subject may be already subjected to a treatment, by any chemotherapeutic agent, or may be untreated yet.

The term ‘DLBCL subject’ refers to a subject having DLBCL originating from a population of DLBCL subjects, from early to late stage of DLBCL, the said subjects undergoing or not undergoing a therapeutic treatment, and in particular DLBCL subjects experiencing relapsing DLBCL.

The ‘iron-score’ according to the invention is a GEP (Gene Expression Profile)-based iron-score; it is defined by the sum of the beta coefficients of the Cox model for each prognostic gene, weighted by ±1 according to the patient signal above or below the probe set Maxstat value.

By ‘prognosis marker’, it means a marker relevant to assess the outcome of the subject. In particular the expression profile or expression level of 1 to 11 genes and/or proteins identified in the present invention as being differentially expressed in B-Cell Lymphoma subjects, represents a prognosis marker that permits to identify subjects having ‘good prognosis’ from subjects having ‘bad prognosis’.

The 11 genes for DLBCL identified to be informative to assess the outcome of the subject are also named in the disclosure as ‘genes of interest’ or ‘prognosis genes’ or ‘prognostic genes’.

By ‘good prognosis’ or ‘good outcome’ according to the present invention, it means the survival of the subject.

By ‘poor prognosis’ or ‘poor outcome’ according to the present invention, it means the ‘disease relapse’ or the ‘death’ of the subject.

By ‘therapeutic treatment targeting iron metabolism’ according to the invention, it encompasses iron chelators and small molecules that sequester lysosomal iron. Examples of such molecules are illustrated later in the disclosure.

The term “treating” or “treatment” means stabilizing, alleviating, curing, or reducing the progression of the B-Cell Lymphoma, in particular DLBCL.

A ‘biological sample’ according to the invention refers to a biological sample obtained, isolated or collected from a subject, in particular a cell culture, a cell line, a tissue biopsy or a fluid such as a blood or bone marrow. In particular, the biological sample is a tissue biopsy comprising lymph nodes or spleen or a fluid comprising lymphocytes B like blood or bone marrow.

By a “reference sample”, it is meant a biological sample of a patient whose clinical outcome is known (i.e. the duration of the disease-free survival (DFS), or the event free survival (EFS) or the overall survival (OS) or both). Preferably, a pool of reference samples comprises at least one (preferably several, more preferably at least 5, more preferably at least 6, at least 7, at least 8, at least 9, at least 10) ‘good outcome’ patient(s) and at least one (preferably several, more preferably at least 6, at least 7, at least 8, at least 9, at least 10) ‘bad outcome’ patient(s). The highest the number of reference samples, the better for the reliability of the method of prediction of the outcome of the subject tested according to the invention.

Said reference samples (collection samples of B-Cell Lymphoma subjects) for which expression profile of the prognosis genes is evaluated, permits to measure predetermined reference values (PREV and PREL as further disclosed), which are used for comparison purposes.

### BRIEF DESCRIPTION OF THE FIGURES

FIG. 1: Prognostic value of the Iron-score in DLBCL patients.

Patients of the Lenz R-CHOP cohort (n=233) were ranked according to increased iron score and a maximum difference in OS (overall survival) was obtained with iron score of −0.16872 (also named ‘cut point’) splitting patients into high-risk and low-risk groups (A) Iron score also had a prognostic value in three other independent cohorts of patients (Melnick R-CHOP cohort, Lenz CHOP cohort and FFPE R-CHOP cohort) (B, C and D).

Iron score is significantly higher in ABC (activated B cell-DLBCL subtype) compared to GCB (germinal center cell-DLBCL subtype) DLBCL patients. (E).

FIG. 2: Genes involved in iron homeostasis presenting a prognostic value in patients with DLBCL.

FIG. 2 A presents the schematic model of the study and selection of 11 prognosis genes. High expression of three genes was associated with a good prognosis including ALAS1, HIF1A, and LRP2 (FIG. 2 B). At the opposite, high expression of eight genes was associated with a poor prognosis: HMOX1, HMOX2, HFE, ISCA1, SLC25A37, PPOX, STEAP1 and TMPRSS6 (FIG. 2 C).

FIG. 3: Iron chelators and Ironomycin inhibit DLBCL cell growth DLBCL cells were incubated with different concentrations of iron chelators or vehicle for 96H. Inhibitory concentration 50% (IC₅₀) was calculated with concentration-response curve after treatment with Deferoxamin (A), Deferasirox, (B) and Ironomycin (C). Cell viability was examined using quantification of ATP assay. Data are expressed as mean percentage+/−SEM of at least three independent experiments performed in sixplicate. IC₅₀ are summarized in the table 6.

FIG. 4: Ironomycin induced G1/S cell cycle arrest and DNA damage.

Cells were incubated with vehicle, with 7 μM Deferoxamine, 80 μM Deferasirox or with IC₅₀ Ironomycin for 72 hours. Cell cycle was analyzed using flow cytometry, S phase was stained by an anti-BrdU antibody after BrdU incorporation and DNA content was strained by 4′,6-diamidino-2-phenylindole (DAPI) for OCI-LY3 (A) and DB cells (B). Histograms represent the mean percentage and SEM of each cell cycle phase of three independent experiments. * and ** indicate a significant difference of P<0.05 and P<0.01, respectively with paired student t-test.

Cells were incubated with vehicle or with 7 μM Deferoxamine, 80 μM Deferasirox or 50 nM Ironomycin for 24 hours. DSB induction was monitored by Phospho-H2A·X (Ser139) staining and nuclei were stained with DAPI. Foci were visualized by fluorescence microscopy. Original magnification X 63. Scale bar: 10 μm. The percentage of cells with more than phospho-H2A·X (Ser139) foci per cell (±SEM) is displayed in the histograms. ** and **** indicate a significant difference of P<0.01 and P<0.0001, respectively with Fisher's exact test, NS: non-significant (C).

Cells were treated with iron chelators (80 μM Deferasirox), and Ironomycin (20 nM and 100 nM) during 24H. Protein levels of Phospho-H2A·X (S139) were analyzed by western blot (D)

DB and OCI-LY3 DLBCL derived cell-line was treated with 50 nM Ironomycin during 24H. Immunofluoresence staining for foci of phospho-RPA2 T21, S phase (EdU integration) and DAPI was performed in DLBCL cells. (Magnification x63). Scale bar: 10 μm. The percentage of cells with more than 10 phospho-RPA2 T21 foci per cell is displayed in the histograms (E).

At least 300 cells were counted for each group. Statistical difference was tested using a Fisher's exact test. * P<0.05, ** P<0.01, *** P<0.001, **** P<0.0001. NS: non-significant.

FIG. 5: Ironomycin induces defective DNA replication fork progression.

DB and OCI-LY3 cells were labeled with IdU, treated with vehicle, gemcitabine (10 μM) or Ironomycin (100 μM) and labeled with CIdU (30 minutes each), and then harvested for DNA fiber assay (A and B).

DB cells were pretreated or not by deoxynucleotides for 30 minutes, labeled with IdU, treated with vehicle, gemcitabine (10 μM) or Ironomycin (100 μM) and labeled with CIdU (30 minutes each), and then harvested for DNA fiber assay (C and D).

Boxplot represent median±interquartile ranges and dots represent outliers of track length (expressed in μm). Results represent at least 200 track measurements. * P<0.05, **** P<0.0001, NS: non-significant, Mann-Whitney test.

FIG. 6: Ironomycin presents a significant toxicity on DLBCL primary cells and potentializes doxorubicin cytotoxicity.

The DB DLBCL-derived cell lines were treated with increasing concentrations of Ironomycin combined with Doxorubicin for 96 h and cell viability was tested by ATP quantification to obtain the viability matrix. The synergy matrix was calculated as described in Material and Methods (A).

Primary DLBCL cell were treated with Ironomycin and/or Doxorubicin and incubated during 96H with CD40 L. Tumorous cells (B) were analyzed by flow cytometry (described in material and method) and expressed in % of control.

Hematopoietic progenitor colony-forming units assay were performed with CD34+ cells from apheresis of 5 donors. Cells were cultured in hydroxyl-methyl-cellulose medium with or without conventional chemotherapy or Ironomycin. CFU-C, CFU-E and CFU-GM were counted after 14 days culture (C).

Results represent the median±IQR of each population cells of five patients. Statistical significance was tested using t-test of pairs: * P<0.05, ** P<0.01 *** P<0.001, **** P<0.0001 and NS: non-significant.

FIG. 7: Ironomycin induces mortality of primary DLBCL cells from patients.

Primary samples of DLBCL patients were cultured with their microenvironment recombinant CD40L in presence or absence of 50 nM and 100 nM of Ironomycin. Percentage of malignant DLBCL cells for each individual patients were analyzed by flow cytometry and expressed in % of control (A). Table presenting the patients characteristics (B). The effect of Ironomycin alone or in combination with Doxorubicin on normal CD3+ cells was assessed by flow cytometry (C).

FIG. 8: Synergistic effect of Ironomycin with Venetoclax (Bcl2 inhibitor)

Three DLBCL-derived cell lines were treated with increasing concentrations of Ironomycin combined with Venetoclax for 96 h and cell viability was tested by ATP quantification to obtain the viability matrix. The synergy matrix was calculated as described in Material and Methods. FIG. 8A: U2932 cell line; FIG. 8B: DB cell line; FIG. 8C: OCI-LY3 cell line.

FIG. 9: Synergistic effect of Ironomycin with Ibrutinib (BTK inhibitor)

Two DLBCL-derived cell lines were treated with increasing concentrations of Ironomycin combined with Ibrutinib for 96 h and cell viability was tested by ATP quantification to obtain the viability matrix. The synergy matrix was calculated as described in Material and Methods. FIG. 9A: U2932 cell line; FIG. 8B: DB cell line.

FIG. 10: Synergistic effect of Ironomycin with Entospletinib (Syk inhibitor)

Two DLBCL-derived cell lines were treated with increasing concentrations of Ironomycin combined with Venetoclax for 96 h and cell viability was tested by ATP quantification to obtain the viability matrix. The synergy matrix was calculated as described in Material and Methods. FIG. 10A: U2932 cell line; FIG. 10B: DB cell line.

FIG. 11: Synergistic effect of Ironomycin with Idelalisib (PI3K inhibitor)

Three DLBCL-derived cell lines were treated with increasing concentrations of Ironomycin combined with Venetoclax for 96 h and cell viability was tested by ATP quantification to obtain the viability matrix. The synergy matrix was calculated as described in Material and Methods. FIG. 11A: U2932 cell line; FIG. 11B: DB cell line; FIG. 11C: OCI-LY3 cell line.

### DETAILED DESCRIPTION OF EMBODIMENTS OF THE INVENTION

The inventors have identified a set of 11 genes and/or proteins involved in the iron metabolism, which are differentially expressed in individuals having DLBCL (DLBCL cells) as compared to healthy subjects (normal B cells). This gene expression profile (GEP)-based risk score may be advantageously used for identifying subjects with poor outcome that may benefit of a targeted treatment (also named personalized medicine) comprising an iron inhibitor. A score value has been calculated, taking into account the beta coefficient for each gene or protein, based on the Cox statistical model.

As illustrated in the examples, the inventors identified, from a list of 62 genes involved in the regulation of iron biology and using Maxstat R function and Benjamini Hochberg multiple testing correction, 11 genes demonstrated a prognostic value in two independent cohorts of DLBCL patients (n=233 and n=181 respectively).

In particular, the inventors demonstrated that:
high expression of three genes was associated with a good prognosis (‘good outcome’) including ALAS1 (Aminolevulinate, delta-synthase 1), HIF1A (Hypoxia inducible factor 1, alpha subunit), and LRP2 (Low density lipoprotein-related protein 2); and
high expression of eight genes was associated with a poor prognosis (‘poor outcome’): HMOX1 (Heme oxygenase (decycling) 1), HMOX2 (Heme oxygenase (decycling) 2), HFE (Hemochromatosis), ISCA1 (Iron-sulfur cluster assembly 1 homolog), SLC25A37 (Solute carrier family 25 (mitochondrial iron transporter), member 37) also named MSCP (Mitochondrial solute carrier protein), PPOX (Protoporphyrinogen oxidase), STEAP1 (Six transmembrane epithelial antigen of the prostate 1) and TMPRSS6 (Transmembrane protease, serine 6).

So the present invention concerns the use of an iron-score based on the expression level of at least 2 genes, in particular at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, as a prognosis marker in subjects having B-cell Lymphoma in particular DLBCL, in particular for identifying subjects with a poor outcome such as a relapse and/or death.

Such B-Cell lymphoma subjects, in particular DLBCL subjects with a poor outcome such as a relapse and/or death identified according to the invention by their iron-score value, may be advantageously treated by a targeted therapeutic treatment comprising an inhibitor of iron metabolism.

In a particular embodiment, the said targeted therapeutic treatment comprises a molecule targeting iron metabolism in particular iron chelator or small molecule sequestering lysosomal iron, in particular selected in the group consisting of Deferasirox, Deferoxamine, Deferiprone, Salinomycin, analogs or derivatives thereof, preferably salinomycin and nitrogen containing salinomycin derivatives.

By ‘molecule targeting iron metabolism’ according to the invention, it means in particular iron chelators and small molecules sequestering lysosomal iron. Iron chelators are small molecules susceptible to interact reversibly with iron. And small molecules sequestering lysosomal iron are loose iron binders that accumulate in the endosomal/lysosomal compartment able to block the metal in this organelle. Examples of such compounds are disclosed later in the description.

By ‘derivatives thereof’ according to the invention, it means synthetic small molecules chemically derived from salinomycin exhibiting a more potent activity and potentially lower toxicity against healthy cells.

By ‘at least 1, in particular at least 5’ genes and/or proteins, it means 1, 2, 3, 4, in particular 5, 6, 7, 8, 9, 10, 11, 12, 13, 14, 15 genes, or 5, 6, 7, 8, 9, 10, 11, 12, 13, 14, 15 proteins.

In an embodiment, the combination of 2 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1 is evaluated.

In an embodiment, the combination of 3 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1 is evaluated.

In an embodiment, the combination of 4 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1 is evaluated.

In an embodiment, the combination of 5 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1, is evaluated.

In another embodiment, the combination of 6 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1, is evaluated.

In another embodiment, the combination of 7 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1, is evaluated.

In another embodiment, the combination of 8 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1, is evaluated.

In another embodiment, the combination of 9 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1 is evaluated.

In another embodiment, the combination of 10 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1 is evaluated.

In another and preferred embodiment, the combination of 11 genes and/or proteins encoded by the said genes selected in the group consisting of HMOX2, PPOX, TMPRSS6, HFE, SLC25A37, STEAP1, ISCA1, HMOX1, LRP2, HIF1A, and ALAS1 is evaluated.

The NCBI references for each gene are mentioned in the table 1 hereunder:

TABLE 1

| | | Ref Seq | Ref Seq | |
|---|---|---|---|---|
| Gene | | Transcript ID | Protein ID | |
| symbol | Gene Name | (NCBI) | (NCBI) | Gene ID |
| HMOX2 | Heme oxygenase (decycling) 2 | NM_001127204 | NP_001120676 | 3163 |
| PPOX | Protoporphyrinogen oxidase | NM_000309 | NP_000300 | 5498 |
| TMPRSS6 | Transmembrane protease, | NM_001289000 | NP_001275929 | 164656 |
| | serine 6 | | | |
| HFE | Hemochromatosis | NM_000410 | NP_000401 | 3077 |
| SLC25A37 | Solute carrier family 25 | NM_016612 | NP_057696 | 51312 |
| or MSCP | (mitochondrial iron transporter), | | | |
| | member 37 or Mitochondrial solute | | | |
| | carrier protein | | | |
| STEAP1 | Six transmembrane epithelial | NM_012449 | NP_036581 | 26872 |
| | antigen of the prostate 1 | | | |
| ISCA1 | Iron-sulfur cluster assembly | NM_030940 | NP_112202 | 81689 |
| | 1 homolog | | | |
| HMOX1 | Heme oxygenase (decycling) 1 | NM_002133 | NP_002124 | 3162 |
| LRP2 | Low density lipoprotein- | NM_004525 | NP_004516 | 4036 |
| | related protein 2 | | | |
| HIF1A | Hypoxia inducible factor 1, | NM_001243084 | NP_001230013 | 3091 |
| | alpha subunit | | | |
| ALAS1 | Aminolevulinate, delta-synthase 1 | NM_000688 | NP_000679 | 211 |

Expression Level of the Set of the Said Genes or Proteins of Interest (‘Prognosis Genes’)

Such measures are made in vitro, starting from a subject's sample, and necessary involve transformation of the sample. Indeed, no measure of a specific gene expression level can be made without some type of transformation of the sample. Most technologies rely on the use of reagents specifically binding to the RNA of interest, thus resulting in a modified sample further including the detection reagent. In addition, most technologies also involve some preliminary extraction of RNA from the subject's sample before binding to a specific reagent. The claimed method may thus also comprise a preliminary step of extracting RNA from the subject's sample.

The expression level of the set of genes and/or proteins, in particular selected in the group consisting of HMOX2, PPOX, TMPRSS6, HFE, SLC25A37, STEAP1, ISCA1, HMOX1, LRP2, HIF1A, and ALAS1, according to the invention, may be measured by any techniques commonly used.

The presence or level of said genes is determined by usual method known from man skilled in the art. In particular, each gene expression level may be measured at the genomic and/or nucleic and/or protein level. In a preferred embodiment, the expression profile is determined by measuring the amount of nucleic acid transcripts of each gene, such as PCR, quantitative PCR (qPCR), NGS (Next-Generation Sequencing (NGS)) and RNA sequencing. In another embodiment, the expression profile is determined by measuring the amount of protein produced by each of the genes.

The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, next generation sequencing and hybridization with a labelled probe.

PCR primers for the DNA amplicons encompassing the genes of interest disclosed above were designed using the genomic sequence obtained from the NCBI.

In particular, the level of mRNA expression for each of the genes of the set may be performed by the well-known techniques of the skilled in the art such as hybridization technique and/or amplification technique (PCR), using suitable primers or probes that are specific for each of the genes mRNA.

Illustratively, mRNA may be extracted, for example using lytic enzymes or chemical solutions or extracted by commercially available nucleic-acid-binding resins following the manufacturer's instructions. Extracted mRNA may be subsequently detected by hybridization, such as Northern blot, and/or amplification, such as quantitative or semiquantitative RT-PCR. Other methods of amplification include ligase chain reaction (LCR), transcription-mediated amplification (TMA), strand displacement amplification (SDA) and nucleic acid sequence based amplification (NASBA). In some embodiments, the level of mRNA expression for each of the genes of interest may be measured by the mean of quantification of the cDNA synthesized from said mRNA, as a template, by one reverse transcriptase.

The amount of mRNA can be measured by any technology known by a person skilled in the art, including mRNA microarrays, quantitative PCR, next generation sequencing and hybridization with a labelled probe. In particular, real time quantitative RT-PCR (qRT-PCR) may be useful. In some embodiments, qRT-PCR can be used for both the detection and quantification of RNA targets. Commercially available qRT-PCR based methods (e.g., Taqman® Array) may for instance be employed, the design of primers and/or probe being easily made based on the sequence of ‘prognostic genes’ disclosed above. mRNA assays or arrays can also be used to assess the levels of the mRNAs in a sample.

In some embodiments, mRNA oligonucleotide array can be prepared or purchased. An array typically contains a solid support and at least one oligonucleotide contacting the support, where the oligonucleotide corresponds to at least a portion of a mRNA.

Any suitable assay platform can be used to determine the presence of the mRNA in a sample. For example, an assay may be in the form of a membrane, a chip, a disk, a test strip, a filter, a microsphere, a multiwell plate, and the like. An assay system may have a solid support on which an oligonucleotide corresponding to the mRNA is attached. The solid support may comprise, for example, a plastic, silicon, a metal, a resin, or a glass. The assay components can be prepared and packaged together as a kit for detecting an mRNA. To determine the expression profile of a target nucleic sample, said sample is labelled, contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the person skilled in the art.

Methods for determining the quantity of mRNA by microarrays or by RNA sequencing may also be used. In certain embodiments, complexes between the double-stranded nucleic acids resulting from amplification and fluorescent SYBR® molecules may be obtained and then the fluorescence signal generated by the SYBR® molecules complexed with the said amplified nucleic acids may be measured. Identification of suitable primers that are specific for each of the genes mRNA consists of a routine work for the one skilled in the art.

In a particular embodiment and as illustrated in the examples for DLBCL subjects, the method for determining the quantity of mRNA by microarrays uses probesets for the specific 11 prognostic genes disclosed above. Mention may be made of the Affymetrix HG-U133 plus 2.0 microarrays and probesets ID related to said specific 11 prognostic genes. In a particular embodiment, method for determining the quantity of mRNA by microarrays uses 12 probesets for the specific 11 prognostic genes (including 2 probesets for 1 gene), as illustrated in the further examples.

In some embodiments, detection by hybridization may be performed with a detectable lable, such as fluorescent probes, enzymatic reactions or other ligands (eg avidin/biotin).

The presence or level of said proteins may be measured by well-known techniques including detection and quantification of the protein of interest by the means of any type of ligand molecule that specifically binds thereto, including nucleic acids (for example nucleic acids selected for binding through the well-known SELEX method), antibodies and antibody fragments. The antibodies to said given protein of interest may be easily obtained with the conventional techniques, including generation of antibody-producing hybridomas.

Thus, in preferred embodiments, expression of a marker is assessed using for example:
a radio-labelled antibody, in particular, a radioactive moiety suitable for the invention may for example be selected within the group comprising 3H, 1211, 1231, 14C or 32P;
a chromophore-labelled or a fluorophore-labelled antibody, wherein a luminescent marker, and in particular a fluorescent marker, suitable for the invention may be any marker commonly used in the field such as fluorescein, fluorescent probes, coumarin and its derivatives, phycoerythrin and its derivatives, or fluorescent proteins such as GFP or the DsRed;
a polymer-backbone-antibody;
an enzyme-labelled antibody, said labelling enzyme suitable for the invention may be an alkaline phosphatase, a tyrosinase, a peroxydase, or a glucosidase; for example, suitable avidin-labelled enzyme may be an avidin-Horse Radish Peroxydase (HRP), and a suitable substrate may be AEC, 5-bromo-4-chloro-3-indolyl phosphate (BCIP), nitro blue tetrazolium chloride (NBT);
an antibody derivative, for example an antibody conjugated with a substrate or with the protein or ligand of a protein-ligand pair, in particular a biotin, a streptavidin or an antibody binding the polyhistidine tag;
an antibody fragment, for example a single-chain antibody, an isolated antibody hypervariable domain, etc., which binds specifically to a marker protein or a fragment thereof, including a marker protein which has undergone all or a portion of its normal post-translational modification.

In a particular and preferred embodiment, expression of a marker is assessed using a GFP fluorescent protein.

In vitro techniques for detection of a biological marker protein include enzyme linked immunosorbent assays (ELISAs), Western blots, immunoprecipitations and immunofluorescence.

In a particular and preferred embodiment, the preferred in vitro methods for detecting and quantifying level expression of said genes of interest according to the present invention, include micro-arrays, NGS, RNA sequencing and PCR techniques.

Calculation of a Score Value (‘Iron Score’) from Said Expression Level of Genes or Proteins of Interest

The score value or ‘prognosis score’ or ‘iron score’ according to the invention, based on the expression level of the ‘prognosis genes’ as defined above, will help classifying the B-Cell lymphoma subjects as having a ‘good outcome’ or a ‘bad outcome’.

The lower the expression of genes related to ‘bad outcome’ is, the better for the subject's survival. Therefore, the higher the level of iron score is, the more likely the subject is to respond to a treatment targeting iron metabolism. In a preferred embodiment, the subject may thus be predicted as having ‘poor outcome’ and consequently being likely to respond to a treatment targeting iron metabolism based on comparison of the expression level of said prognosis genes in the patient's sample with one or more threshold value(s) (predetermined reference value, PREV).

In a particular embodiment, the patient is considered as having poor outcome, when the iron score is higher than a threshold value. Such a threshold value may be determined based on a pool of reference samples, as defined above. In this embodiment, patients are classified into two groups based on said expression level of prognosis genes, depending if this expression level is lower or greater than said threshold value. Patients with iron score higher than the threshold value are considered as having a poor outcome and likely to respond to treatment targeting iron metabolism.

In another embodiment, the method further comprises determining a prognostic score based on the expression level of said prognosis genes, wherein the prognostic score indicates whether the patient has a poor outcome. In particular, said prognosis score may indicate whether the patient is likely to have a poor outcome or a bad outcome if it is higher or lower than a predetermined threshold value (PREV or PREL) (dichotomized result).

As a result, a prognosis score may be determined based on the analysis of the correlation between the expression level of said prognosis genes of the invention and progression free survival (PFS) or overall survival (OS) of a pool of reference samples, as defined above. A PFS and/or OS score, which is a function correlating PFS or OS to the expression level of said prognosis genes of the invention, may thus be used as prognosis score for prediction of the outcome of the subject.

The expression level for each combination of the 11 genes and/or proteins of interest as disclosed above according to the invention, may be associated with a score value, also named ‘iron-score’ in the present invention.

Following the measurement of the expression level of at least 2, in particular at least 5 or more genes and/or proteins encoded by the said 5 or more genes selected in a group consisting of HMOX2, PPOX, TMPRSS6, HFE, SLC25A37, STEAP1, ISCA1, HMOX1, LRP2, HIF1A, and ALAS1, in a biological sample obtained from a B-Cell lymphoma, in particular DLBCL subject (step a) of the method), the computation of a score value may be performed by a method comprising the following steps:
i) comparing the expression level determined at step a) with a predetermined reference expression level (PREL);
ii) calculating the score value (‘iron score’) with the following formula:

$$Score = {\sum }_{i = 1}^{n} β i \times Ci$$

wherein
n represents the number of genes and/or protein which expression level is measured, i.e. n being comprised from 1 to 11, in particular from 5 to 11,
βi represents the regression β coefficient reference value for a given gene or protein, and
Ci represents “1” if the expression level of said gene or protein is higher than the predetermined reference level (PREL) or Ci represents “−1” if the expression level of the gene or the protein is lower than or equal to the predetermined reference level (PREL).

The predetermined reference level (PREL) is often referred as to “maxstat value” or “maxstat cutpoint”.

In some embodiments, a good prognosis status or ‘good outcome’ refers to an individual having a score value lower than or equal to a predetermined reference value (PRV).

In some embodiments, a bad prognosis status or ‘bad outcome’ refers to an individual having a score value higher than a predetermined reference value (PRV).

The “regression β coefficient reference value” may be easily determined by the skilled man in the art for each gene or protein using the well-known statistical Cox model, which is based on a modelling approach to analyze survival data. The purpose of the model is to simultaneously explore the effects of several variables on survival. When it is used to analyze the survival of patients in a clinical trial, the model allows isolating the effects of the treatment from the effects of other variables. The Cox model may also be referred as to proportional hazards regression analysis. In particular, this model is a regression analysis of the survival times (or more specifically, the so-called “hazard function”) with respect to defined variables. The “hazard function” is the probability that an individual will experience an event, e.g. death, within a small time interval, given that the individual has survived up to the beginning of the interval. It can therefore be interpreted as the risk of dying at time t. The quantity h0 (t) is the baseline or underlying hazard function and corresponds to the probability of dying (or reaching an event) when all the defined variables are zero. The baseline hazard function is analogous to the intercept in ordinary regression (since exp0=1). The “regression coefficient β” gives the proportional change that can be expected in the hazard, related to changes in the defined variables. The coefficient β is estimated by a statistical method called maximum likelihood. In survival analysis, the hazard ratio (HR) (Hazard Ratio=exp(β)) is the ratio of the hazard rates corresponding to the conditions described by two sets of defined variables.

Predetermined reference values, such as PREL or PRV, which are used for comparison purposes may consist of “cut-off” values.

For example, each reference (“cut-off”) value PREL for each gene or protein may be determined by carrying out a method comprising the following steps:
a) providing a collection of samples from subjects (patients) suffering from B-Cell lymphoma, in particular DLBCL (‘reference samples’);
b) determining the expression level of the relevant gene or protein for each sample contained in the collection provided at step a);
c) ranking the samples according to said expression level;
d) classifying said samples in pairs of subsets of increasing, respectively decreasing, number of members ranked according to their expression level;
e) providing, for each sample provided at step a), information relating to the actual clinical outcome for the corresponding B-Cell lymphoma patient, in particular the DLBCL patient (i.e. the duration of the disease-free survival (DFS), or the event free survival (EFS) or the overall survival (OS) or both);
f) for each pair of subsets of tumor tissue samples, obtaining a Kaplan Meier percentage of survival curve;
g) for each pair of subsets of tumor tissue samples calculating the statistical significance (p value) between both subsets;
h) selecting as reference value PREL for the expression level, the value of expression level for which the p value is the smallest.

As an illustration, the expression level of a gene or a protein of interest may be assessed for 100 samples (‘reference samples’) of 100 subjects (patients). The 100 samples are ranked according to the expression level of said given gene or protein. Sample 1 may have the highest expression level and sample 100 may have the lowest expression level. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding DLBCL patient, Kaplan Meier curves may be prepared for each of the 99 groups of two subsets. Also for each of the 99 groups, the p value between both subsets was calculated. The reference value PREL is then selected such as the discrimination based on the criterion of the minimum p value is the strongest. In other words, the expression level corresponding to the boundary between both subsets for which the p value is minimum is considered as the reference value. It should be noted that according to the experiments made by the inventors, the reference value PREL is not necessarily the median value of expression levels.

The skilled in the art also understands that the same technique of assessment of the PRV could be used for obtaining the reference value and thereafter for assessment of the response to the targeted treatment of the present invention comprising an inhibitor of iron metabolism. However in one embodiment, the reference value PRV is the median value of PRV.

As illustrated further in the examples of the invention, the prognostic information of these 11 genes of interest (‘prognosis genes’) was then combined in a GEP (Gene Expression Profile)-based iron-score. The ‘iron score’ is defined by the sum of the beta coefficients of the Cox model for each prognostic gene, weighted by ±1 according to the patient signal above or below the probe set Maxstat value as previously described (Herviou et al., 2018). Maxstat algorithm segregated the Lenz R-CHOP cohort into two groups with 39.5% of the patients with an iron score≥−0.168 and 60.5% of the patients with a iron score 5-0.168 with a maximum difference in overall survival (OS). Patients with high-risk iron score have a median OS of 50.6 months versus not reached for patients with low iron score (P=3,7E-15)) in the Lenz R-CHOP cohort. As illustrated in the examples, the prognostic value of the iron score was validated in the three additional independent cohorts for OS.

As further disclosed in the examples of the invention, the Cox univariate and multivariate analyses of overall survival (OS) in patients with diffuse large B-cell lymphoma (R-CHOP Lenz cohort, n=233) were made, using prognostic poor outcome-related factors, such as germinal-center B-cell-like subgroup (GCB subtype) or activated B cell-like subtype (ABC subtype), age and the IPI (international prognostic index). The said prognostic factors were tested individually (A), two by two (B), or in multivariate analysis (all variables), using a Cox regression model. As shown in the examples further illustrated, based on the P-values and hazard ratios (HR) of each condition, comparing with other poor outcome-related factors, such as GCB or ABC subtype, age and the IPI in multivariate COX analysis, the iron score remained an independent prognostic factor. Altogether, these data highlight a deregulation of iron metabolism genes in association with a poor outcome in DLBCL.

In particular embodiment, the regression β coefficient reference value, the hazard ratio and the reference value PREP for each of the 11 genes or proteins of interest were measured. These values were measured on references samples of DLBCL subjects (>200 samples) but may vary from 5 to 15% depending of the number of reference samples. The highest the number of reference samples, the better for the reliability of the method of prediction of the outcome of the subject tested according to the invention.

The Table 2 below illustrates relevant parameter ranges for Maxstat_Cutpoint, beta coefficient and Hazard ratio (HR) for each of the 11 genes of interest.

TABLE 2

| | | | Hazard |
|---|---|---|---|
| | | | ratio |
| Name | Maxstat_Cutpoint | beta coef | (HR) |
| ALAS1 | 2117/2587 | −0.49/−0.4 | 0.36 |
| HMOX1 | 2679/3275 | 0.21/0.25 | 1.7 |
| HIF1A | 20566/25136 | −0.46/−0.38 | 0.38 |
| HMOX2 | 830/1015 | 0.42/0.51 | 2.9 |
| TMPRSS6 | 421/514 | 0.34/0.42 | 2.4 |
| STEAP1 | 167/204 | 0.29/0.35 | 2.1 |
| LRP2 | 65/79 | −0.31/−0.26 | 0.52 |
| SLC25A37 | 896/1095 | 0.31/0.38 | 2.2 |
| PPOX | 669/818 | 0.37/0.46 | 2.6 |
| SLC25A37 | 339/415 | 0.23/0.28 | 2.2 |
| ISCA1 | 1051/1284 | 0.25/0.31 | 1.9 |
| HFE | 129/158 | 0.31/0.38 | 2.2 |

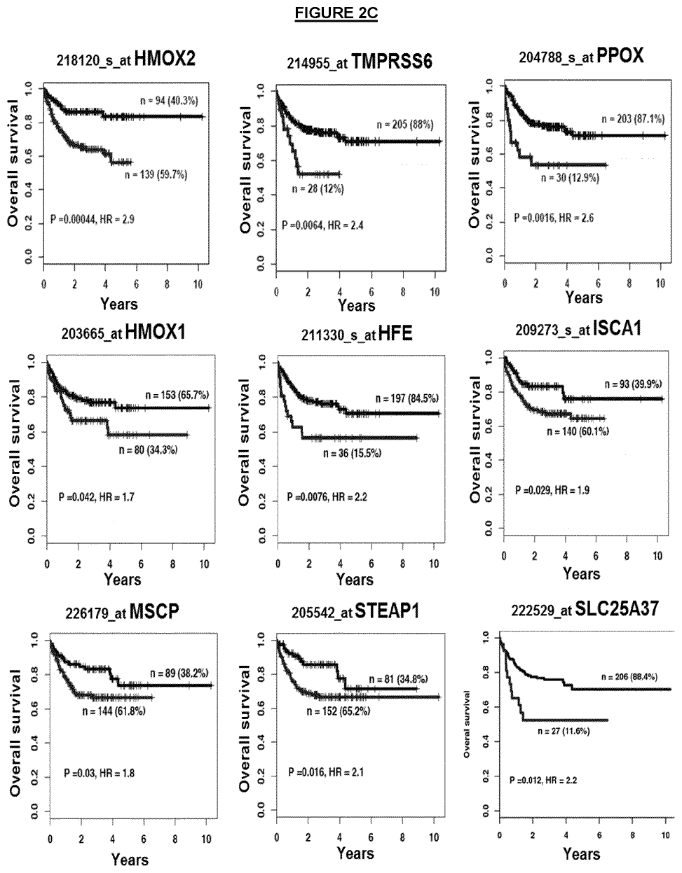

This table 2 and related FIGS. 2B and 2C show that the genes HMOX2, PPOX, TMPRSS6, SLC25A37, HFE, STEAP1 have the higher Hazard ratio (HR>2), meaning that an iron score based on the expression level of at least 2, 3, 4 or 5 of these genes would be a good prognostic marker for DLBCL patients.

The score may be generated by a computer program and may be used in the in vitro method according to the invention in particular for identifying a DLBCL subject with a poor outcome that may benefit of a targeted treatment comprising an inhibitor of iron metabolism, and/or for further monitoring the efficacy of a targeted therapeutic treatment.

Method for Identifying a B-Cell Lymphoma Subject with a Poor Outcome, in Particular DLBCL Subject with a Poor Outcome

The present invention also concerns an in vitro method for identifying a B-Cell Lymphoma subject with a poor outcome, in particular a DLBCL subject with a poor outcome that may benefit from a targeted therapeutic treatment comprising an inhibitor of iron metabolism, comprising the steps of:
a) Measuring the expression level of at least 2, in particular at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject;
b) Calculating a score value from said expression level obtained at step a)
c) Classifying and identifying the said subject with a poor outcome according to the score value in comparison to a predetermined reference value.

In a particular embodiment, the present invention concerns an in vitro method for identifying a DLBCL subject with a poor outcome that may benefit from a targeted therapeutic treatment comprising an inhibitor of iron metabolism, comprising the steps of:
a) Measuring the expression level of at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject;
b) Calculating a score value from said expression level obtained at step a)
c) Classifying and identifying the said subject with a poor outcome according to the score value in comparison to a predetermined reference value.

The expression level of the said genes or proteins of interest at step a) are measured according to the detection and/or quantification methods well known in the art. Examples of such methods are disclosed above.

The calculation of the score value (‘iron score’) at step b) is made as disclosed above, in particular by:
i) comparing the expression level determined at step a) with a predetermined reference expression level (PREL);
ii) calculating the score value with the following formula:

$$Score = {\sum }_{i = 1}^{n} β i \times Ci$$

wherein
n represents the number of genes and/or protein which expression level is measured, i.e. n being comprised from 5 to 11,
βi represents the regression β coefficient reference value for a given gene or protein, and
Ci represents “1” if the expression level of said gene or protein is higher than the predetermined reference level (PREL) or Ci represents “−1” if the expression level of the gene or the protein is lower than or equal to the predetermined reference level (PREL).

The classification of the subject according to ‘good outcome’ subgroup and ‘bad outcome’ subgroup is based according to its iron-score value in comparison to a predetermined reference value (PRV).

In the present invention, a subject with a ‘poor outcome’ refers to an individual having a score value higher than a predetermined reference value (PRV).

In a particular embodiment, for DLBCL subjects, when the iron score is based on the expression level of the 11 genes or proteins consisting of HMOX2, PPOX, TMPRSS6, HFE, SLC25A37, STEAP1, ISCA1, HMOX1, LRP2, HIF1A, and ALAS1, the predetermined reference value (PRV) or ‘cutpoint’ is −0,16872, meaning that in the step c) of the in vitro method described above, the subject with a poor outcome according to the iron score are the ones having an iron score value higher than −0,16872.

Method for Monitoring the Efficacy of a Targeted Therapeutic Treatment

Another object of the invention is an in vitro method for monitoring the efficacy of a therapeutic treatment targeting iron metabolism in a subject having a high-risk B-Cell lymphoma, in particular Diffuse Large B-Cell Lymphoma (DLBCL) and undergoing said treatment, comprising the steps of:
a) Measuring the expression level of at least 2, in particular at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T1 before or during or after the subject has been administered said therapeutic treatment targeting iron metabolism;
b) Calculating a first score value at time T1 from said expression level obtained at step a),
c) Measuring the expression level of at least 2, in particular at least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T2 before or during or after the subject has been administered the said therapeutic treatment targeting iron metabolism, wherein said time T2 is posterior to said time T1;
d) Calculating a second score value at time T2 from said expression level obtained at step c),
e) Assessing the efficacy of a therapeutic treatment based on the comparison the second score value at T2 obtained at step d) with the first score value at T1 obtained at step b).

The expression level of genes or proteins of interest according to the invention at step a) and d) are made as disclosed above.

The first and second score values (iron-score values), respectively at time T1 and time T2, are made as disclosed above.

In a preferred embodiment, the invention concerns an in vitro method for monitoring the efficacy of a therapeutic treatment targeting iron metabolism in a subject having a high-risk Diffuse Large B-Cell Lymphoma (DLBCL) and undergoing said treatment, comprising the steps of:
a) Measuring the expression level of the 11 genes or proteins consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T1 before the subject has been administered said therapeutic treatment comprising an active agent against DLBCL and/or an inhibitor of iron metabolism;
b) Calculating a first score value at time T1 from said expression level obtained at step a),
c) Measuring the expression level of the 11 genes or proteins consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject at a time T2 after the subject has been administered the said therapeutic treatment comprising an active agent against DLBCL and/or an inhibitor of iron metabolism, wherein said time T2 is posterior to said time T1;
d) Calculating a second score value at time T2 from said expression level obtained at step c),
e) Assessing the efficacy of a therapeutic treatment based on the comparison the second score value at T2 obtained at step d) with the first score value at T1 obtained at step b).

Kits Dedicated for In Vitro Methods of the Invention

The kits of the invention are dedicated for in vitro methods of the invention.

By “dedicated”, it is meant that reagents for the determination of an expression level of genes and/or proteins as identified above in the kit of the invention essentially consist of reagents for determining the expression level of the above (i) expression profiles, optionally with one or more housekeeping gene(s), and thus comprise a minimum of reagents for determining the expression of other genes than those mentioned in above described (i) expression profiles and housekeeping genes. For instance, a dedicated kit of the invention preferably comprises no more than 20, preferably no more than 12, preferably no more than 10, preferably no more than 9, 8, 7, 6, 5, 4, 3, 2, or 1 reagent(s) for determining the expression level of a gene that does not belong to one of the above described (i) expression profiles and that is not a housekeeping gene.

Such a kit may further comprise instructions for determination of poor or good outcome of the subject.

So the present invention relates to a kit dedicated to in vitro methods of the invention, in particular for determining whether a B-Cell Lymphoma subject, in particular a DLBCL subject, has a high risk of death and/or relapse, comprising or consisting of reagents for determining the expression level of at least 2, preferably at least 5, more preferably at least 10 and even preferably at least 11 genes and/or proteins selected in the group consisting of HMOX2, PPOX, TMPRSS6, HFE, SLC25A37, STEAP1, ISCA1, HMOX1, LRP2, HIF1A, and ALAS1 in a sample of said subject, and no more than 20, preferably no more than 12, preferably no more than 10, preferably no more than 9, 8, 7, 6, 5, 4, 3, 2, or 1 reagent(s) for determining the expression level of a gene that does not belong to one of the above described.

Reagents for determining the expression level of said prognostic genes in a sample of said subject, may notably comprise or consist of primers pairs (forward and reverse primers) and/or probes (in particular labeled probes, comprising a nucleic acid specific for the target sequence and a label attached thereto, in particular a fluorescent label) specific for said prognostic genes or a microarray comprising a sequence specific for said prognostic genes.

The design of primers and/or probe can be easily made by those skilled in the art based on the sequences of said genes disclosed above.

In a particular embodiment, said kits comprise specific amplification primers and/or probes for the specific quantitative amplification of transcripts of ‘prognosis genes’ identified above and/or a nucleic microarray for the detection of said ‘prognosis genes’ identified above.

The present invention also relates to a kit dedicated to in vitro methods of the present invention comprising a set of primers and/or probes for measuring the expression level of at the least 5, preferably at least 10, and even preferably 11 genes and/or proteins encoded by the said at least 5, preferably at least 10, and even preferably 11 genes selected in the group consisting of HMOX2, PPOX, TMPRSS6, HFE, SLC25A37, STEAP1, ISCA1, HMOX1, LRP2, HIF1A, and ALAS1, as a set of prognostic markers for performing an in vitro methods as disclosed above. In particular, the said kit comprises no more than 20, preferably no more than 12, preferably no more than 10, preferably no more than 9, 8, 7, 6, 5, 4, 3, 2, or 1 reagent(s) for determining the expression level of a gene that does not belong to one of the above described.

In a first embodiment, the kit of the present invention is used for performing an in vitro method for identifying a B-Cell Lymphoma subject with a poor outcome, in particular a DLBCL subject with a poor outcome that may benefit from a targeted therapeutic treatment as disclosed above.

In another embodiment, the kit of the present invention is used for performing an in vitro method for monitoring the efficacy of a therapeutic treatment targeting iron metabolism in a subject having a high-risk B-Cell Lymphoma, in particular Diffuse Large B-Cell Lymphoma (DLBCL) and undergoing said treatment.

The kits for detection of poor outcome B-Cell lymphoma, in particular DLBCL patients or respectively for monitoring the efficacy of a targeted therapeutic treatment, may also comprises all reagents needed for the detection and/or quantification of expression of the said genes or proteins of interest according to the invention.

In a particular embodiment, the kit dedicated to DLBCL subjects comprises a set of probe sets for measuring the expression level of 11 genes and/or proteins encoded by the said 11 genes selected in the group consisting of HMOX2, PPOX, TMPRSS6, HFE, SLC25A37, STEAP1, ISCA1, HMOX1, LRP2, HIF1A, and ALAS1. In particular, the said kit comprises no more than 20, preferably no more than 12, preferably no more than 10, preferably no more than 9, 8, 7, 6, 5, 4, 3, 2, or 1 reagent(s) for determining the expression level of a gene that does not belong to one of the above described. The kit may also comprise generic reagents useful for the determination of the expression level of any gene, such as Taq polymerase or an amplification buffer.

Pharmaceutical Composition

Another object of the invention is a pharmaceutical composition comprising, in a pharmaceutical acceptable vehicle, a molecule targeting iron metabolism, in particular an iron chelator or small molecule sequestering lysosomal iron, in particular selected in the group consisting of Deferasirox, Deferoxamine, Deferiprone, Salinomycin, analogs or derivatives thereof, preferably salinomycin and nitrogen-containing analogs of salinomycin, for use in a method for treating B-Cell lymphoma subjects, in particular Diffuse large B-cell lymphoma (DLBCL), Follicular lymphoma, Marginal zone B-cell lymphoma (MZL) or Mucosa-Associated Lymphatic Tissue lymphoma (MALT), Small lymphocytic lymphoma (also known as chronic lymphocytic leukemia, CLL), and Mantle cell lymphoma (MCL), preferably subjects having DLBCL.

In particular, the said pharmaceutical composition is used in a method for treating subjects identified according to the in vitro method of the invention as having a poor outcome according to iron-score and consequently likely to display a B-Cell lymphoma relapse and/or death, preferably a DLBCL relapse and/or death.

Examples of nitrogen-containing analogs of salinomycin are disclosed in the WO2016/038223.

In a particular embodiment, the iron chelator is a nitrogen-containing analog of salinomycin of formula (I)

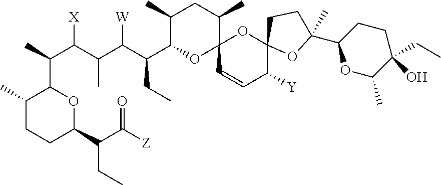

wherein:
W is selected from the group consisting of ═O; —NR₁R₂; —NR₃—(CH₂)ₙ—NR₄R₅; —O—(CH₂)ₙ—NR₄R₅; —NR₃—(CH₂)ₙ-N⁺R₆R₇R₈ and —O—(CH₂)ₙ-N⁺R₆R₇R₈;
X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ-N⁺R₆R₇R₈ and —O—(CH₂)ₙ-N⁺R₆R₇R₈,
Y is selected from the group consisting of —OH; ═N—OH; —NR₁R₂; —NR₃—(CH₂)ₙ—NR₄R₅; —O—(CH₂)ₙ—NR₄R₅; —NR₃—(CH₂)ₙ-N⁺R₆R₇R₈ and —O—(CH₂)ₙ-N⁺R₆R₇R₈, R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl; R₃ is selected from the group consisting of H; (C₁-C₆)-alkyl; (C₁-C₆)-alkyl-aryl; R₄ and R₅, identical or different, are selected from the group consisting of H; (C₁-C₆)-alkyl; aryl and (C₁-C₆)-alkyl-aryl;
R₆, R₇ and Ra, identical or different, are selected from the group consisting of (C₁-C₆)-alkyl; aryl and (C₁-C₆)-alkyl-aryl;
Z is a group such as OH; NHNR₉R₁₀; NHOC(O)R₁₁; N(OH)—C(O)R₁₁; OOH, SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
R₁₂ is selected from the group consisting of H; (C₁-C₁₆)-alkyl; (C₃-C₁₆)-alkenyl;
(C₃-C₁₆)-alkynyl; aryl; heteroaryl; (C₁-C₆)-alkyl-aryl; (C₁-C₆)-alkyl-heteroaryl n=0, 2, 3, 4, 5 or 6, with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ-N⁺R₆R₇R₈ and —O—(CH₂)ₙ-N⁺R₆R₇R₈.

Advantageously, R₁ and R₂, identical or different, are selected from the group consisting of H; (C₁-C₁₆)-alkyl, advantageously (C₃-C₁₄)-alkyl, more advantageously (C₃-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

Advantageously, R₁ and R₂ are not both H.

More advantageously, R₁ is H and R₂ is selected from the group consisting of (C₁-C₁₆)-alkyl, advantageously (C₃-C₁₄)-alkyl, more advantageously (C₃-C₁₄)-alkyl; (C₃-C₁₆)-alkenyl, advantageously (C₃-C₅)-alkenyl; (C₃-C₁₆)-alkynyl, advantageously (C₃-C₅)-alkynyl; (C₃-C₁₆)-cycloalkyl, advantageously (C₃-C₆)-cycloalkyl; (C₁-C₆)-alkyl-aryl, advantageously benzyl, and (C₁-C₆)-alkyl-heteroaryl, advantageously CH₂-pyridynyl.

Advantageously, R₃ is selected from the group consisting of H and (C₁-C₆)-alkyl. Preferably, R₃ is H.

Advantageously, Z is OH, OOH, NHNH₂, NHOH, or NH₂OH, preferably OH.

In a preferred embodiment, the iron chelator is a compound of formula (I) as defined above, wherein X is OH, Z is OH and Y is NR₁R₂ where R₁ is H and R₂ is selected from the group consisting of (C₁-C₁₆)-alkyl, advantageously (C₈-C₁₄)-alkyl; (C₃-C₁₆)-alkenyl, advantageously (C₃-C₅)-alkenyl; (C₃-C₁₆)-alkynyl, advantageously (C₃-C₅)-alkynyl and (C₃-C₁₆)-cycloalkyl, advantageously (C₃-C₆)-cycloalkyl; C₁-C₆)-alkyl-aryl, advantageously benzyl, and (C₁-C₆)-alkyl-heteroaryl, advantageously CH₂-pyridynyl.

In a more preferred embodiment, the iron chelator is a compound of formula (I) as defined above, wherein W is =O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl.

The compound of formula (I) wherein W is =O, X is OH, Z is OH, and Y is NR₁R₂ where R₁ is H and R₂ is a (C₃-C₅)-alkynyl group, preferably propargyl, is also named Ironomycin or compound AM5 as disclosed in the patent application WO2016/038223.

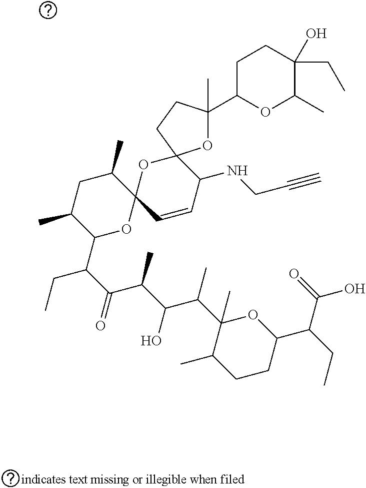

The compound of formula (I) wherein W is =O, X is OH, Z is OH, and Y is NR₁R₂ where R₁ is H and R₂ is a (C₃-C₆)-cycloalkyl group, preferably cyclopropyl, is also named AM23 as disclosed in the patent application WO2016/038223.

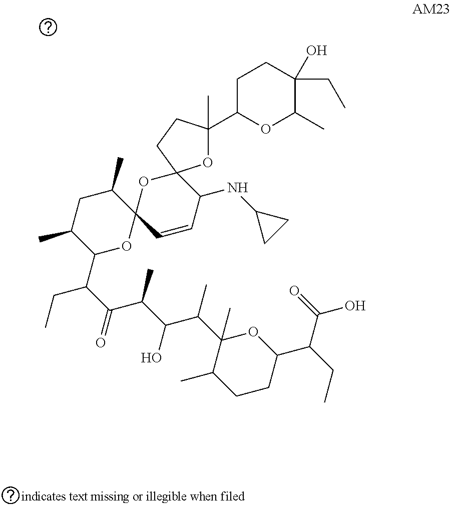

In another particular embodiment, W is =O, X is OH, Z is OH, and Y is NR₁R₂ where R₁ is H and R₂ is a (C₃-C₆)-cycloalkyl group, in particular a substituted cyclopropyl as disclosed hereunder:

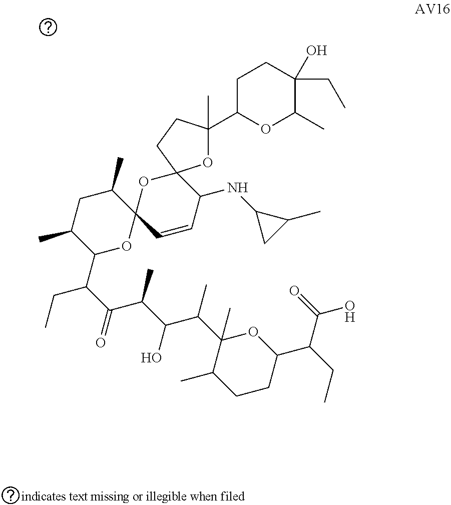

In another particular embodiment, W is =O, X is OH, Z is OH, and Y is NR₁R₂ where R₁ is H and R₂ is a (C₁-C₆)-alkyl-aryl group, in particular a benzyl group substituted by an hydroxy, as disclosed hereunder:

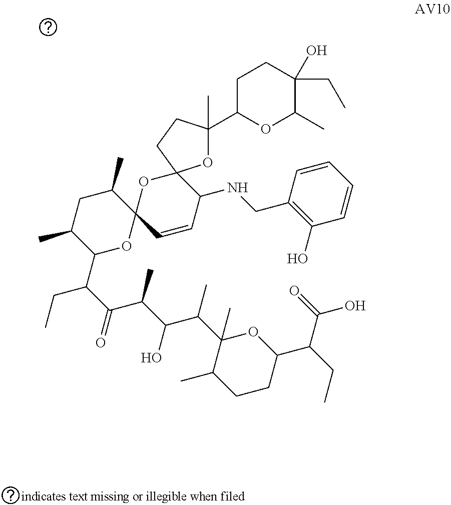

In another particular embodiment, W is =O, X is OH, Z is OH, and Y is NR₁R₂ where R₁ is H and R₂ is a (C₁-C₆)-alkyl-pyridyl group, in particular a CH₂-pyridinyl group, as disclosed hereunder:

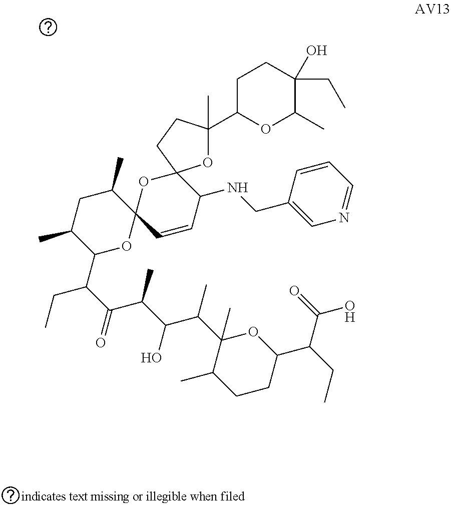

The compounds AM5, AM23, AV10, AV13 and AV16, preferably AM5 are particular and preferred compounds used in the pharmaceutical composition, pharmaceutical product and therapeutic uses disclosed hereunder.

The pharmaceutical composition for use according to the invention comprises at least one compound of formula (I) as defined above, a pharmaceutical salt, solvate or hydrate thereof, and at least one pharmaceutically acceptable excipient.

For the purpose of the invention, the term ‘pharmaceutically acceptable’ is intended to mean what is useful to the preparation of a pharmaceutical composition, and what is generally safe and non-toxic, for a pharmaceutical use.

The term<<pharmaceutically acceptable salt, hydrate of solvate>> is intended to mean, in the present invention, a salt of a compound which is pharmaceutically acceptable, as defined above, and which possesses the pharmacological activity of the corresponding compound.

Such salts comprise:
hydrates and solvates,
acid addition salts formed with inorganic acids such as hydrochloric, hydrobromic, sulfuric, nitric and phosphoric acid and the like; or formed with organic acids such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphthalenesulfonic, propionic, succinic, dibenzoyl-L-tartaric, tartaric, p-toluenesulfonic, trimethylacetic, and trifluoroacetic acid and the like, and
salts formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate and sodium hydroxide.

The pharmaceutical compositions for use according to the invention can be intended to oral, sublingual, subcutaneous, intramuscular, intravenous, transdermal, topical or rectal administration. The active ingredient can be administered in unit forms for administration, mixed with conventional pharmaceutical carriers, to animals or to humans. When a solid composition is prepared in the form of tablets, the main active ingredient is mixed with a pharmaceutical vehicle and other conventional excipients known to those skilled in the art.

The compounds of the invention can be used in a pharmaceutical composition at a dose ranging from 0.01 mg to 1000 mg a day, administered in only one dose once a day or in several doses along the day, for example twice a day. The daily administered dose is advantageously comprised between 5 mg and 500 mg, and more advantageously between 10 mg and 200 mg. However, it can be necessary to use doses out of these ranges, which could be noticed by the person skilled in the art.

The invention also concerns a method for treating a B-Cell lymphoma subject, in particular DLBCL subjects, preferably having a poor outcome as identified by the in vitro method of the invention, more preferably a DLBCL subject having a poor outcome as identified by the in vitro method of the invention, which method comprises (i) determining whether the subject is likely to have a relapse and/or death, by the in vitro method according to the invention and based on iron score, and (ii) administering a molecule targeting iron metabolism to said subject if the subject has been determined to have a ‘poor outcome’.

The method may further comprise, if the subject has been determined to be unlikely to have a ‘poor outcome’ a step (iii) of administering an alternative anticancer treatment to the subject Such alternative anticancer treatment depends on the specific B-Cell lymphoma and on previously tested treatments, but may notably be selected from radiotherapy, other chemotherapeutic molecules, or other biologics such as monoclonal antibodies directed to other antigens.

In certain embodiments, an anti-B cell lymphoma treatment may include a treatment with anticancer compounds, radiation, surgery or stem cell transplant.

Pharmaceutical Product (Also Named “Combination Product”)

The present invention also relates to a pharmaceutical product comprising:
(i) a molecule targeting iron metabolism, in particular an iron chelator or a small molecule sequestering lysosomal iron and
(ii) another anti-cancer agent selected from the group consisting of agents used either in chemotherapy, in targeted treatments, in immune therapies, and in combinations thereof, as combination product for simultaneous, separate or staggered use as a medicament in the treatment of B-Cell lymphoma, in particular DLBCL, in particular in DLBCL subjects with a poor outcome according to in vitro method of the invention.

By ‘agents used in chemotherapy’ according to the invention, it means drugs also named ‘chemo drugs’ able to stop the growth of cancer cells, either by killing the cells or by stopping them from dividing.

By ‘agents used in targeted treatments’ according to the invention, it means drugs or other substances able to identify and attack specific types of cancer cells with less harm to normal cells. Some targeted therapies block the action of certain enzymes, proteins, or other molecules involved in the growth and spread of cancer cells. Other types of targeted therapies help the immune system kill cancer cells or deliver toxic substances directly to cancer cells and kill them. Targeted therapy may have fewer side effects than other types of cancer treatment. Most targeted therapies are either small molecule drugs or monoclonal antibodies.

By ‘agents used in immune therapies’ according to the invention, it means substances also named ‘immunomodulatory agents’ able to stimulate or suppress the immune system to help the body fight cancer. Some types of immunotherapy only target certain cells of the immune system. Others affect the immune system in a general way. Types of immunotherapy include as examples cytokines, and some monoclonal antibodies.

In some embodiments, anticancer compounds may include a chemo drug, in particular selected in a group comprising vincristine, cyclophosphamide, etoposide, doxorubicin, liposomal doxorubicin, cytarabine, melphalan, Bendamustine, Cisplatin, daunorubicin, Fludarabine, Methotrexate.

In some embodiments, anticancer compounds may include:
Bcl2 inhibitors, or
PI3K inhibitors, or
BTK inhibitors, or
Syk inhibitors
   and mixtures thereof.

‘Bcl-2 (B-cell lymphoma 2) inhibitors’ are a class of compounds that inhibit Bcl-2 family of regulator proteins that regulate cell death (apoptosis), by either inhibiting (anti-apoptotic) or inducing (pro-apoptotic) apoptosis. They are used to selectively induce apoptosis in malignant cells. Mention may be made of ABT-737 and navitoclax (ABT-263), and preferably Venetoclax (ABT-199, CAS No. 1257044-40-8) that is a highly selective inhibitor, which inhibits Bcl-2, but not Bcl-xL or Bcl-w.

‘Phosphoinositide 3-kinase (PI3K) inhibitors’ are a class of compounds that inhibit one or more of the phosphoinositide 3-kinase enzymes. These enzymes form part of the PI3K/AKT/mTOR pathway, which is a pathway involved in cell growth and survival, as well as several other processes that are frequently activated in many cancers. By inhibiting these enzymes, PI3K inhibitors cause cell death, inhibit the proliferation of malignant cells, and interfere with several signaling pathways. Mention may be made of Idelalisib (CAL-101, GS-1101, CAS No. 870281-82-6).

‘Bruton's tyrosine kinase’(abbreviated Btk or BTK)’ inhibitors, also known as tyrosine-protein kinase BTK inhibitors, are a class of compounds that inhibit a tyrosine kinase that plays a crucial role in B cell development. Mention may be made of Ibrutinib (PCI-32765, CAS No. 936563-96-1). ‘Spleen tyrosine kinase (Syk) inhibitors’ are class of compounds that inhibit Syk, a cytosolic non-receptor protein tyrosine kinase (PTK) that is mainly expressed in hematopoietic cells and was recognized as a critical element in the B-cell receptor signaling pathway. Several oral Syk inhibitors including fostamatinib (R788), entospletinib (GS-9973), cerdulatinib (PRT062070), and TAK-659 are being assessed in clinical trials. Mention may be made in particular to entospletinib (GS-9973, CAS No. 1229208-44-9).

In some embodiments, anticancer compounds may include a proteasome inhibitor, in particular selected in a group comprising bortezomib, carfilzomib and ixazomib.

In some embodiments, the immunomodulatory agent is selected in a group comprising thalidomide, lenalidomide, pomalidomide and a derivative thereof.

In some embodiments, anticancer compounds may include a corticosteroid, in particular selected in a group comprising dexamethasone and prednisone.

In some embodiments, anticancer compounds may include an epidrug including histone deacetylase (HDAC) inhibitor, DNMT inhibitor, EZH2 inhibitor, BET inhibitor, PRMT5 inhibitor, IDH inhibitor.

In some embodiments, anticancer compounds may include a monoclonal antibody, in particular selected in a group comprising Rituximab and obinutuzumab.

In some embodiments, anticancer compounds may include immunotherapy with CAR-T cells, in particular selected in a group comprising Tisagenlecleucel and axicabtagene ciloleucel and lisocabtagene maraleucel

In a particular embodiment, for treatment of DLBCL subjects, the molecule targeting iron metabolism, in particular an iron chelator or a small molecule sequestering lysosomal iron (i) is selected in the group consisting of Deferasirox, Deferoxamine, Deferiprone, Salinomycin, analogs or derivatives thereof, preferably salinomycin and nitrogen-containing analogs of salinomycin as defined above, and the other anti-cancer agent (ii) is selected from the group consisting of agents used in chemotherapy (iia), in particular cyclophosphamide, doxorubicin, etoposide, Venetoclax, Idelalisib, Ibrutinib, entospletinib and combinations thereof.

In a particular and preferred embodiment for DLBCL treatment, the iron chelator (i) is a compound of formula (I) as defined above, wherein W is =O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl and the other chemotherapy compound (ii) is Doxorubicin, Venetoclax, Idelalisib, Ibrutinib, or entospletinib.

Another preferred subject-matter of the invention for DLBCL treatment is a pharmaceutical product or composition comprising:
(i) a compound of formula (I) as defined above, wherein W is =O, X is OH, Z is OH, and Y is NR₁R₂ where R₁ is H and R₂ is selected from the group consisting of (C₃-C₅)-alkynyl and (C₃-C₆)-cycloalkyl, preferably (C₃-C₅)-alkynyl and
(ii) a chemotherapy compound selected in the group consisting of cyclophosphamide, doxorubicin, etoposide, Venetoclax, Idelalisib, Ibrutinib, entospletinib preferably doxorubicin, Venetoclax, Idelalisib, Ibrutinib, or entospletinib.

The present invention will be now illustrated by the non-limitative examples.

### EXAMPLES

Material and Methods

Gene Expression Data Analyses and Building of the Iron Score

The list of 62 genes involved in the regulation of iron biology was established using previously published data (Miller et al., 2011).

Expression of these genes was interrogated in normal B cells (n=5 centroblasts, n=5 centrocytes) and in DLBCL samples (n=73) using data published by the group of Compagno (Compagno et al., 2009). Affymetrix gene expression data are publicly available via the online Gene Expression Omnibus (http://www.ncbi.nlm.nih.gov/geo/) under accession number GSE12195.

The related 62 genes involved in the iron metabolism are listed in the table 3 hereunder:

TABLE 3

| Gene Symbol | Gene Title |
|---|---|
| FTH1 | ferritin, heavy polypeptide 1 |
| EPAS1 | endothelial PAS domain protein 1 |
| HIF1A | hypoxia inducible factor 1, alpha subunit |
| | (basic helix-loop-helix transcription factor) |
| LTF | lactotransferrin |
| UROS | uroporphyrinogen III synthase |
| HMBS | hydroxymethylbilane synthase |
| FECH | ferrochelatase |
| SLC11A2 | solute carrier family 11 (proton-coupled |
| | divalent metal ion transporter), member 2 |
| ABCB6 | ATP binding cassette subfamily B member 6 |
| | (Langereis blood group) |
| HMOX1 | heme oxygenase 1 |
| HEPH | hephaestin |
| PPOX | protoporphyrinogen oxidase |
| CP | ceruloplasmin (ferroxidase) |
| MTF1 | metal-regulatory transcription factor 1 |
| STEAP1 | six transmembrane epithelial antigen of |
| | the prostate 1 |
| FXN | frataxin |
| ALAS1 | 5′-aminolevulinate synthase 1 |
| LRP2 | LDL receptor related protein 2 |
| ACO1 | aconitase 1, soluble |
| HP /// HPR | haptoglobin /// haptoglobin-related protein |
| TFRC | transferrin receptor |
| UROD | uroporphyrinogen decarboxylase |
| FBXL5 | F-box and leucine-rich repeat protein 5 |
| ISCU | iron-sulfur cluster assembly enzyme |
| ISCA1 | iron-sulfur cluster assembly 1 |
| ABCG2 | ATP binding cassette subfamily G member 2 |
| | (Junior blood group) |
| TFR2 | transferrin receptor 2 |
| HFE | hemochromatosis |
| SLC39A14 | solute carrier family 39 (zinc transporter), |
| | member 14 |
| LCN2 | lipocalin 2 |
| FTL | ferritin, light polypeptide |
| TMPRSS6 | transmembrane protease, serine 6 |
| CIAO1 | cytosolic iron-sulfur assembly component 1 |
| HMOX2 | heme oxygenase 2 |
| STEAP3 | STEAP family member 3, metalloreductase |
| NFS1 | NFS1 cysteine desulfurase |
| ALAD | aminolevulinate dehydratase |
| SLC22A17 | solute carrier family 22, member 17 |
| NARFL | nuclear prelamin A recognition factor-like |
| NFU1 | NFU1 iron-sulfur cluster scaffold |
| HAMP | hepcidin antimicrobial peptide |
| SFXN3 | sideroflexin 3 |
| CYBRD1 | cytochrome b reductase 1 |
| SLC25A37 | solute carrier family 25 (mitochondrial iron |
| | transporter), member 37 |
| FLVCR1 | feline leukemia virus subgroup C cellular |
| | receptor 1 |
| SLC40A1 | solute carrier family 40 (iron-regulated |
| | transporter), member 1 |
| SLC25A28 | solute carrier family 25 (mitochondrial |
| | iron transporter), member 28 |
| MON1A | MON1 secretory trafficking family member A |
| SFXN4 | sideroflexin 4 |
| STEAP2 | STEAP family member 2, metalloreductase |
| IREB2 | iron responsive element binding protein 2 |
| STEAP4 | STEAP family member 4 |
| ISCA2 | iron-sulfur cluster assembly 2 |
| SFXN2 | sideroflexin 2 |
| HFE2 | hemochromatosis type 2 (juvenile) |
| SFXN1 | sideroflexin 1 |
| SFXN5 | sideroflexin 5 |
| SCARA5 | scavenger receptor class A, member 5 |
| APEX2 | APEX nuclease (apurinic/apyrimidinic |
| | endonuclease) 2 |
| HPX | hemopexin |
| HIF1AN | hypoxia inducible factor 1, alpha subunit |
| | inhibitor |

Significance analysis of microarray analysis was applied to the 62 selected probe sets in the different samples with 1000 permutations, a fold change of two and a false discovery rate of 0% (t-test).

Gene expression microarray data from two independent cohorts of patients diagnosed with DLBCL and treated by R-CHOP (Rituximab with cyclophosphamide, doxorubicine, vincristine, and prednisone) were used. The first cohort comprised 233 patients (Lenz et al., 2008) and the second one comprised 69 patients (Shaknovich et al., 2010). Pre-treatment clinical characteristics of patients were previously published by the groups of G. Lenz and of R. Shaknovich. Affymetrix gene expression data are publicly available via the online Gene Expression Omnibus (http://www.ncbi.nlm.nih.qov/qeo/) under accession number GSE10846 and GSE23501. They were performed using Affymetrix HG-U133 plus 2.0 microarrays for the two cohorts of patients. The data were analyzed with Microarray Suite version 5.0 (MAS 5.0), using Affymetrix default analysis settings and global scaling as normalization method. The trimmed mean target intensity of each array was arbitrarily set to 500.

In each cohort, the statistical significance of overall survival (OS) of the expression of each probe set of the iron list was calculated by the log-rank test. Multivariate analysis was performed using the Cox proportional hazards model. Survival curves were plotted using the Kaplan-Meier method in the platform Genomicscape (Kassambara et al., 2015). Probe sets with a common prognosis value in the two cohorts were selected. To gather their prognostic information within one parameter, the Iron Score of DLBCL was built as the sum of the beta coefficients weighted by ±1 according to the patient signal above or below the probe set Maxstat value (Kassambara et al., 2012).

Human DLBCL Cell Lines

The 16 DLBCL cell lines (U2932, OCI-LY-3, NU-DHL-1, OCI-LY-19, DB, SUDHL4, OCILY1, SUDHL5, DOHH2, SUDHL10, HT, RI-1, SU-DHL-6, NUDUL-1, WSU-DLCL-2 and OCI-LY-7) were purchased from the DSMZ (Leibniz-Institut DSMZ—Deutsche Sammlung von Mikroorganismen und Zellkulturen GmbH, Germany). They were maintained in RPMI-1640 (Gibco, Invitrogen), supplemented with 10% fetal bovine serum (PAA laboratory GmbH) for U2932, SUDHL-4, HT, DOHH2, SUDHL-10, RI-1, WSU-DLCL-2 cell lines, 20% FBS OCI-LY3, DB, SUDHL-5, NUDHL-1, SU-DHL-6, NUDUL-1 cell lines. OCI-LY1 and OCI-LY7 were cultured in IMDM (Gibco, Invitrogen), supplemented with 20% fetal bovine serum and OCI-LY19 was cultured in MEM alpha modified (Gibco, Invitrogen), supplemented with 20% fetal bovine serum. Cultures were maintained at 37° C. in a humidified atmosphere with 5% CO₂.

Reagents

Deferoxamine (from Novartis Pharma SAS) was dissolved in sterile distilled water and Deferasirox (from Selleckchem S1712), was dissolved in dimethyl sulfoxide (DMSO) to a concentration of 300 mM and 50 mM respectively. Ironomycin also named ‘AM5’ in the patent application WO2016/038223 was dissolved in dimethyl sulfoxide (DMSO) to a concentration of 10 mM. Erastin (from Selleckchem S7242, 10 mM in DMSO) and Ferrostatin-1 (from Selleckchem S7243, 50 mM in DMSO) Q-VD Oph (From SelleckChem S7311, 10 mM in DMSO, 30′ pre-treatment) Iron(III) Chloride Hexahydrate (31232 Sigma Aldrich, 0.1 M in water, 4 h post-treatment), reduced gluthatione GSH (Sigma Aldrich G4251, 0.1 M in PBS) H₂O₂(Sigma Aldrich 216763). Mafosfamide (surrogate of cyclophosphamide, Santa Cruz ChemCruz SC-211761, 10 mM in saline water), Gemcitabine (From Sellekchem S1149, 50 mM in saline water), Doxorubicin (From Sellekchem S1208, 20 mM in DMSO), Venetoclax (From Selleckchem S8048, 10 mM in DMSO), Idelalisib (From Selleckchem S2226, 50 mM in DMSO), Ibrutinib (From Selleckchem S2680, 50 mM in DMSO), entospletinib (From Selleckchem S7523, 50 mM in DMSO), CIdU (abcam ab213715, 20 mM in water), IdU (abcam ab142581, 2 mM in water).

Cell Viability Assay

DLBCL-derived cell lines were cultured for 4 days in 96-well flat-bottom microtiter plates in RPMI 1640 medium, 10% or 20% FCS (control medium) in the presence of various compounds. The number of viable cells in culture was determined using the CellTiter-Glo Luminescent Cell Viability Assay from Promega, Madison, Wis., USA using a Centro LB 960 luminometer (Berthold Technologies, Bad Wildbad, Germany).

This test is based on quantitation of the intracellular ATP present, which signals the presence of metabolically active cells. Data are expressed as the mean percentage of six replicates, normalized to the untreated control.

Primary DLBCL Cells

Lymph node samples were collected after patients' written informed consent in accordance with the Declaration of Helsinki and institutional research board approval from Montpellier University hospital. Cells are obtained from lymph nodes or blood of 5 patients with DLBCL. Cells from blood are obtained by density gradient separation and cells from lymph node are obtained with a tissues dissociator and qualified by Flow cytometry.

Cells are cultured in Gibco® Iscove's MDM (Glutamax) medium (#31980-022) with 20% FBS with antibiotitcs-antimicotics (Gibco Penicillin-streptomycin-amphotericin B 100X, #15240-096) at a density of 0.5×10\hat{ }6 Cell/mL with 50 ng/mL of histidine-tagged CD40L (R&D System, 2706-CL) and 5 μg/mL of anti-histidine antibody R&D System, MAB050), Gibco® pyruvate 100X, #1136-039. Cells are seeded 24H after thawing and treated with various compounds during 72H.

Total cells were counted with trypan bleu and stained with the panel CD45 V500 (BD, #560777), Kappa FITC (Dako, F0434), CD19 PE-Cy7 (BD, #341113), Lambda PE (Dako, R0437), CD3 APC-H7 (BD, #641415), CD10 APC (BD, #332777) and CD20 V450 (BD, #655872) and analyzed by flow cytometry (Canto II cytometer, BD Pharmigen).Tumorous population cells were gated on CD19+, CD45+, CD20+, Kappa or lambda and non-tumorous population cells were gated on CD45+,CD19−, and T cells on CD45+, CD3+, CD19−.

Hematopoietic Progenitor Colony Forming Units (CFU) Assay

Mobilized peripheral blood of donor were collected using an apheresis machine. Cells were CD34+ enriched by immuno-magnetic cell separation and fluorescence activated cell sorting (FACS).

About 250 viable CD34+ cells/mL were cultured in Methocult GF H84434 Medium (Stem Cell, #84434). Using a 3 mL syringe attached to a 16 gauge blunt end needle, 1 mL of the mixture was dispensed into 2 35 mm dishes.

Dishes were incubated at 37° C., in 5% C₀₂, with >95% humidity for 14 days.

Quantification of the Interaction Effect

The interaction between the drugs tested in vitro was investigated with a concentration matrix test, in which increasing concentration of each single drug were assessed with all possible combinations of the other drugs. For each combination, the percentage of expected growing cells in the case of effect independence was calculated according to the Bliss equation (Combes et al., 2019):

fuC=fuA·fuB

where fuC is the expected fraction of cells unaffected by the drug combination in the case of effect independence, and fuA and fuB are the fractions of cells unaffected by treatment A and B, respectively. The difference between the fraction of living cells in the cytotoxicity test and the fuC value was considered as an estimation of the interaction effect, with positive values indicating synergism and negative values antagonism.

Results:

Considering the important role of iron metabolism in cancer cell biology, the inventors first aimed to identify iron metabolism genes associated with a prognostic value in DLBCL. A list of 62 genes involved in the regulation of iron biology was extracted from literature (Miller et al., 2011), as disclosed in the materiel and methods above (table 3).

Using Maxstat R function and Benjamini Hochberg multiple testing correction (Lausen and Schumacher, 1992), 11 genes demonstrated a prognostic value in two independent cohorts of DLBCL patients (n=233 and n=181 respectively) (FIG. 2 A), as disclosed above and reported in the table 4 hereunder:

TABLE 4
Probsets and names of prognostic genes of the Iron score

| Probe set | | |
|---|---|---|
| (Affymetrix HG-U133 | | |
| plus 2.0 microarrays) | Gene | Gene name |
| 205633_s_at | ALAS1 | Aminolevulinate, delta-, |
| | | synthase 1 |
| 203665 at | HMOX1 | Heme oxygenase (decycling) 1 |
| 218120_s_at | HMOX2 | Heme oxygenase (decycling) 2 |
| 211330_s_at | HFE | Hemochromatosis |
| 200989_at | HIF1A | Hypoxia inducible factor 1, |
| | | alpha subunit |
| | | (basic helix-loop-helix |
| | | transcription factor) |
| 209273_s_at | ISCA1 | Iron-sulfur cluster assembly 1 |
| | | homolog (S. cerevisiae) |
| 205710_at | LRP2 | Low density lipoprotein-related |
| | | protein 2 |
| 226179_at | SLC25A37 | Solute carrier family 25 or |
| | or MSCP | Mitochondrial solute carrier protein |
| 204788_s_at | PPOX | Protoporphyrinogen oxidase |
| 222529_at | SLC25A37 | Solute carrier family 25 or |
| | or MSCP | Mitochondrial solute carrier protein |
| 205542_at | STEAP1 | Six transmembrane epithelial |
| | | antigen of the prostate 1 |
| 214955_at | TMPRSS6 | Transmembrane protease, serine 6 |

High expression of three genes was associated with a good prognosis including ALAS1 (Aminolevulinate, delta-synthase 1), HIF1A (Hypoxia inducible factor 1, alpha subunit), and LRP2 (Low density lipoprotein-related protein 2) (FIG. 2B). At the opposite, high expression of eight genes was associated with a poor prognosis: HMOX1 (Heme oxygenase (decycling) 1), HMOX2 (Heme oxygenase (decycling) 2), HFE (Hemochromatosis), ISCA1 (Iron-sulfur cluster assembly 1 homolog), SLC25A37 (Solute carrier family 25 (mitochondrial iron transporter), member 37) also named MSCP (Mitochondrial solute carrier protein), PPOX (Protoporphyrinogen oxidase), STEAP1 (Six transmembrane epithelial antigen of the prostate 1) and TMPRSS6 (Transmembrane protease, serine 6) (FIG. 2 C).

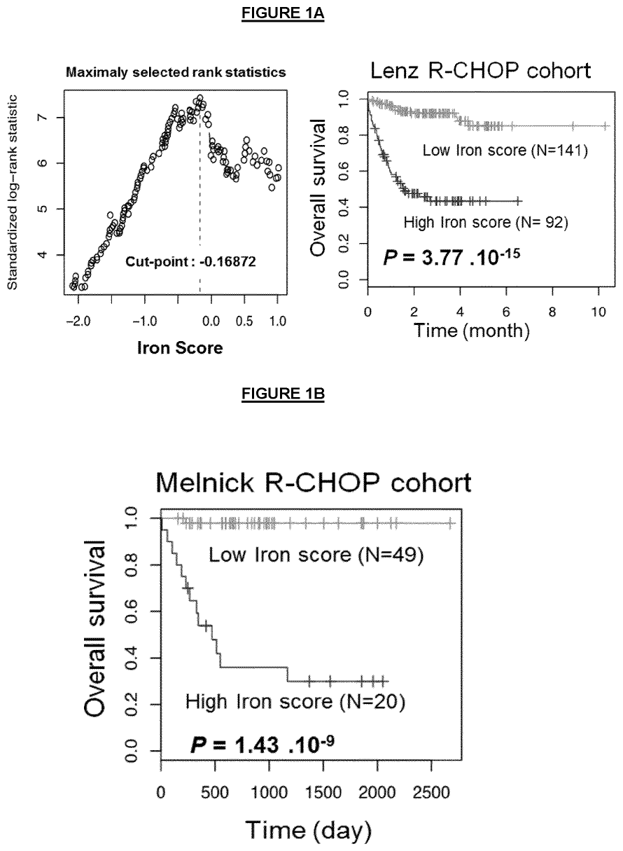

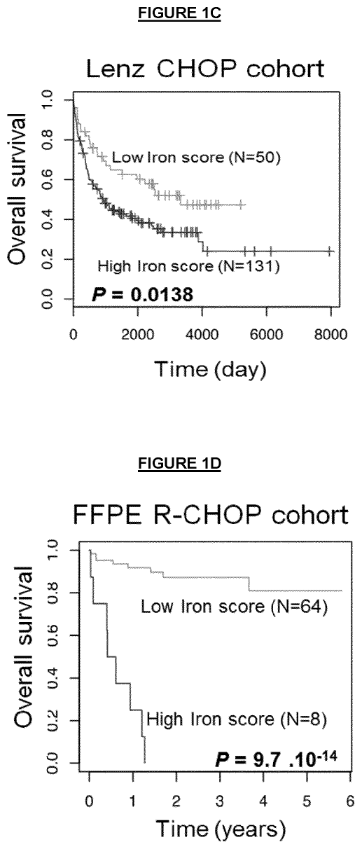

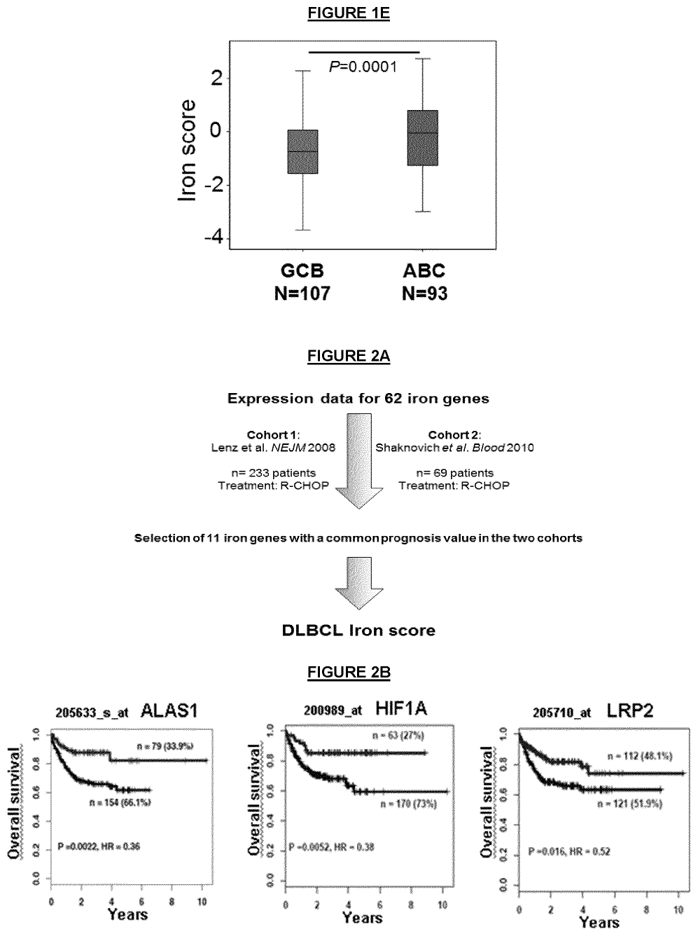

We next combined the prognostic information of these genes in a GEP-based iron-score. The iron score is defined by the sum of the beta coefficients of the Cox model for each prognostic gene, weighted by −1 according to the patient MMC signal above or below the probe set Maxstat value as previously described (Herviou et al., 2018). Maxstat algorithm segregated the Lenz R-CHOP cohort into two groups with 39.5% of the patients with an iron score>−0.168 and 60.5% of the patients with an iron score 5-0.168 with a maximum difference in overall survival (OS; FIG. 1A). Patients with high-risk iron score have a median OS of 50.6 months versus not reached for patients with low iron score (P=3,7E-15)) in the Lenz R-CHOP cohort (FIG. 1A). The prognostic value of the iron score was validated in the three additional independent cohorts for OS (FIGS. 1B, C and D) Cox univariate and multivariate analyses of overall survival (OS) in patients with diffuse large B-cell lymphoma (R-CHOP Lenz cohort, n=233). The indicated prognostic factors were tested individually (A), two by two (B), or in multivariate analysis (all variables) (C) using a Cox regression model. P-values and hazard ratios (HR) are shown in the table 5.

TABLE 5

Overall survival (n = 233)

| Prognostic variable | HR | p value |
|---|---|---|
| A. | | |
| Age (>60 years) | 2.20 | <0.0001 |
| GCB-ABC molecular subgroups | 2.75 | <0.0001 |
| IPI | 1.79 | <0.0001 |
| Iron Score | 4.57 | <0.0001 |
| B. | | |
| Iron Score | 7.74 | <0.0001 |
| Age (>60 years) | 2.19 | 0.005 |
| Iron Score | 6.32 | <0.0001 |
| GCB-ABC molecular subgroups | 2.29 | 0.006 |
| Iron Score | 5.54 | <0.0001 |
| IPI | 1.49 | <0.0001 |
| C. | | |
| Age (>60 years) | 0.92 | NS |
| GCB-ABC molecular subgroups | 2.69 | 0.011 |
| IPI | 1.49 | 0.006 |
| Iron Score | 4.57 | <0.0001 |

NS: not significant at the 5% threshold. The IPI groups were defined as follows: low risk group=IPI score 0 or 1, low-intermediate risk group=IPI score 2, high-intermediate risk group=IPI score 3, and high-risk group=IPI score 4 or 5. IPI, international prognostic index. GCB, germinal-center B-cell-like subgroup. ABC, activated B cell-like subtype.

The Iron score is significantly higher in ABC DLBCL patients compared to GCB (FIG. 1E). Comparing the DNA repair score with other poor outcome-related factors, such as GCB or ABC subtype, age and the IPI in multivariate COX analysis, iron score remained an independent prognostic factor. GSEA analysis revealed that irons score defined high-risk patients are associated with a significant enrichment in MYC target genes and purine metabolism. Iron score defined low-risk DLBCL patients presented a significant enrichment in genes involved in immune response. Altogether, these data highlight a deregulation of iron metabolism genes in association with a poor outcome in DLBCL. Targeting iron metabolism could represent new potential therapeutic avenues for high-risk DLBCL patients.

Targeting Iron Metabolism Induces DLBCL Cell Toxicity

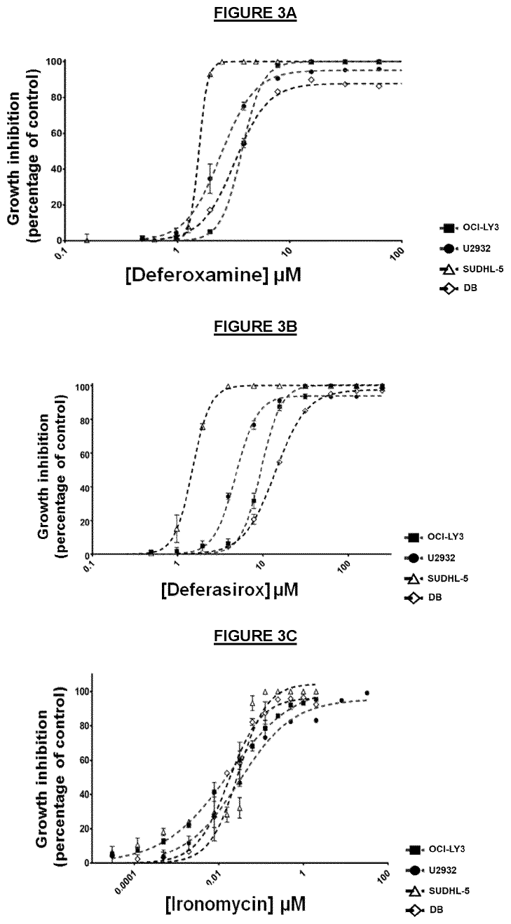

According to these results, we investigated the therapeutic interest of iron chelators Deferoxamine and Deferasirox was using two ABC cell lines OCI-LY3 and U-2932 and two GCB cell lines DB and SUDHL-5. A concentration-dependent reduction in cell viability following treatment with Deferoxamine or Deferasirox was identified. The 50% inhibitory concentration (IC₅₀) values obtained with Deferoxamine were 3.75 μM, 3.24 μM, 2.42 μM and 1.55 μM against OCI-LY3, DB, U2932 and SUDHL5 cells, respectively (FIG. 3 A). And the 50% inhibitory concentration (IC₅₀) values obtained with Deferasirox were 9.50 μM, 13.9 μM, 4.79 μM and 1.48 μM in OCI-LY3, DB, U2932 and SUDHL5 cells, respectively (FIG. 3 B).

TABLE 6
Comparison of 50% inhibitory concentration
(IC₅₀) in μM of iron chelators and Ironomycin

| Cell lines | Deferoxamine | Deferasirox | Ironomycin |
|---|---|---|---|
| OCI-LY3 | 3.7522 | 9.4986 | 0.0168 |
| U2932 | 2.2808 | 4.7940 | 0.0304 |
| SUDHL-5 | 1.5544 | 1.4837 | 0.0289 |
| DB | 3.2481 | 13.9421 | 0.0172 |

Ironomycin is a synthetic derivative of salinomycin that present a therapeutic interest in cancer by accumulating and sequestering iron in lysosomes (Mai et al., 2017). Ironomycin treatment induces a concentration-dependent decrease in cell viability with nanomolar IC₅₀ values compared to Deferoxamine and Deferasirox (16.8 nM, 17.2 nM, 30.4 nM and 28.9 nM in OCI-LY3, DB, U2932 and SUDHL5 cells, respectively) (FIG. 3C).

We extending the analyses to a large panel of 16 DLBCL cell lines. The panel of 16 DLBCL cell lines were incubated with increasing concentrations of Ironomycin or vehicle for 96H. Table 7 hereunder presents the inhibitory concentration 50% (IC₅₀) of Ironomycin for the 16 DLBCL cell-lines. Interestingly, all DLBCL cell lines present an IC₅₀ at nanomolar concentration (range: 7.2-91.5 nM)

TABLE 7

| | | IC₅₀ |
|---|---|---|
| DLBCL derived | | Ironomucin |
| cell line | Sub-group | (μM) |
| SU-DHL-4 | GCB | 0.0072 |
| NU-DUL-1 | ABC | 0.0137 |
| OCI-LY3 | ABC | 0.0168 |
| DB | CGB | 0.0172 |
| OCI-LY1 | GCB | 0.0203 |
| HT | GCB | 0.0241 |
| SU-DHL-5 | GCB | 0.0289 |
| RI-1 | ABC | 0.0302 |
| U-2932 | ABC | 0.0304 |
| WSU-DLCL-2 | GCB | 0.0307 |
| SU-DHL-6 | GCB | 0.0328 |
| SU-DHL-10 | GCB | 0.0365 |
| OCI-LY19 | GCB | 0.0469 |
| NU-DHL-1 | GCB | 0.0505 |
| OCI-LY7 | GCB | 0.0516 |
| DOHH-2 | GCB | 0.0915 |

The inventors further investigated the effect of Iron supplementation on the cell death induced by these treatments. Iron chelators concentrations were chosen according the maximal plasmatic concentration achievable in the patient (Nisbet-Brown et al., 2003). The inventors demonstrated that Iron chelators and Ironomycin induces apoptosis in OCI-LY3 and DB cell lines monitored by Annexin V and PARP cleavage. Iron supplementation significantly inhibited the effect of iron chelators on DLBCL cells apoptosis (P<0.05 and P
<0.01 for Deferoxamine and Deferasirox treatment respectively). However, iron supplementation did not affect ironomycin-induced DLBCL cell cytotoxicity.

The inventors also explored the mechanisms involved in Ironomycin induced DLBCL cell death and showed that:
Ironomycin treatment induces apoptosis in DLBCL cell lines that is partially inhibited by Quinoline-Val-Asp-Difluorophenoxymethylketone (Oph-Q-VD) pan-caspase inhibitor (P<0.001); apoptosis induced by Ironomycin was associated with activation of caspase-3 and −7 and could be rescued by adding Oph-Q-VD;
Ironomycin induces ferroptosis in DLBCL cells underlined by Bodipy-C11 staining indicating the presence of lipid peroxidation. Furthermore, Ironomycin induced cell death could be partially prevented by the ferroptosis inhibitor ferrostatin-1 (Dixon et al., 2012). Ferrostatin did not demonstrated significant toxicity in DLBCL cells but abrogated the effects of erastin, a ferroptosis inductor used as a positive control. Combination of Oph-Q-VD and Fer-1 could not completely abrogate the toxicity mediated ironomycin suggesting that other mechanisms could be involved in ironomycin-induced cell death;
the inventors also investigated if Ironomycin treatment induces autophagy in DLBCL cells. They monitored the expression of the autophagy-associated protein LC31-II (Orhon and Reggiori, 2017) in DLBCL cells following treatment with Ironomycin (100 nM) 24H whereas BIX-01294 was used as positive control (Ding et al., 2013), and identified a clear increase in LC3B II puncta in DLBCL cells upon ironomycin treatment (P<0.0001);
finally, treatment of OCI-LY3 and DB DLBCL cells with iron chelators or ironomycin induces ROS production monitored by fluorescein probe 5-(and-6)-chloromethyl-2′,7′-dichlorodihydrofluorescein diacetate-acetyl ester (CM-H2DCFDA) staining. ROS production mediated by ironomycin was partially inhibited by ferrostin but not by gluthation (GSH). Chaetocin was used as positive control of ROS production inhibited by GSH (Isham et al., 2007).

Taken together, these results demonstrated that Ironomycin induces DLBCL cell toxicity through ferroptosis, autophagy.

Ironomycin Affects DLBCL Cell Division and Induces DNA Damage Response.

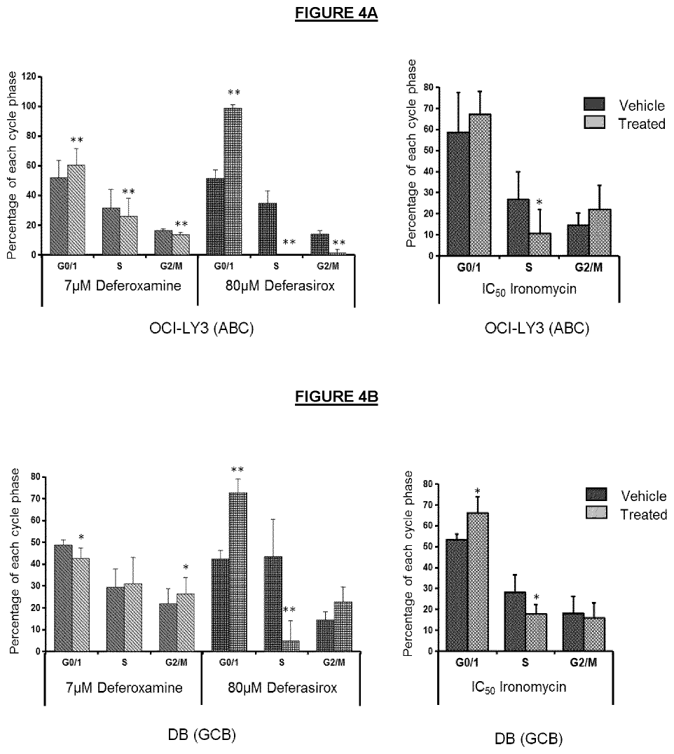

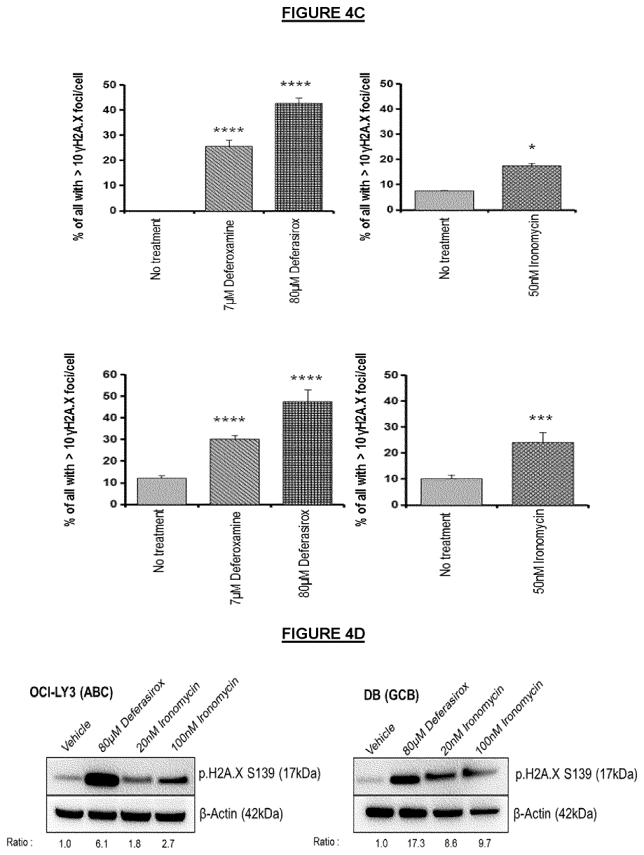

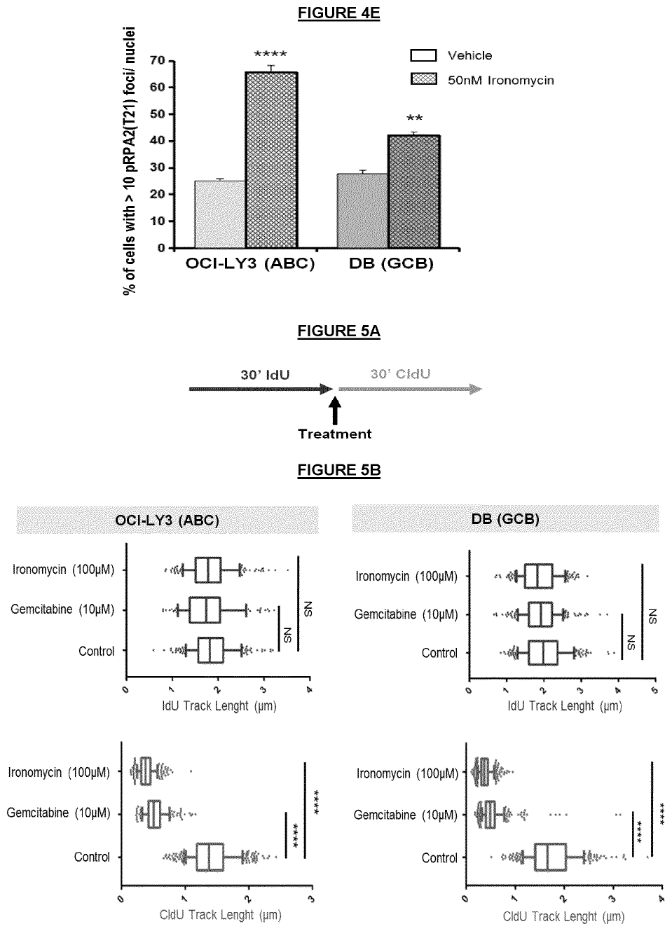

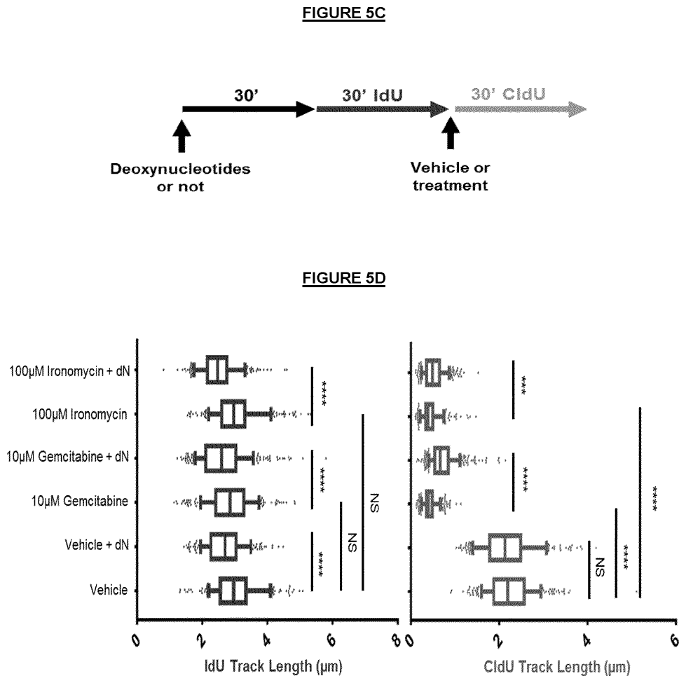

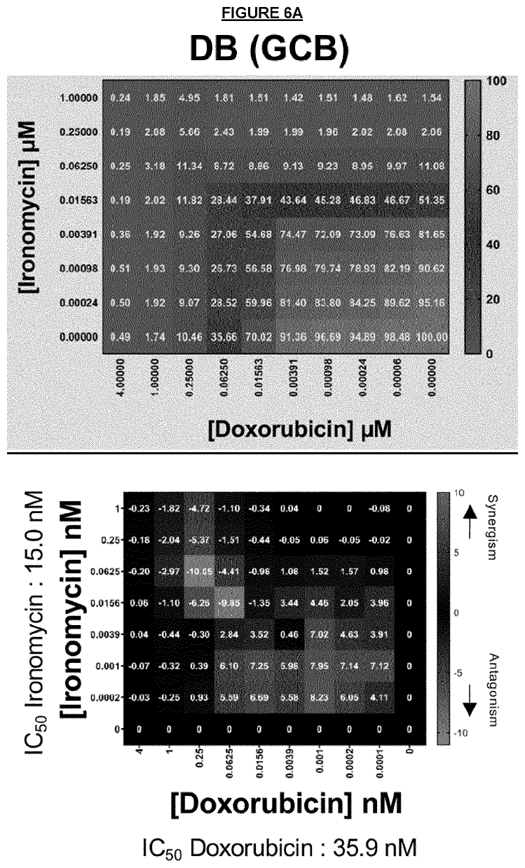

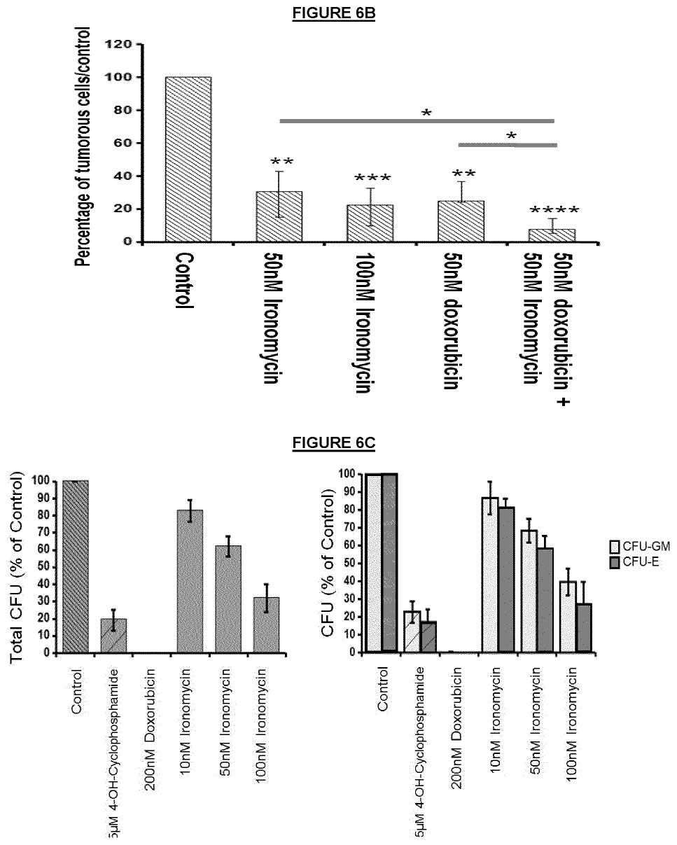

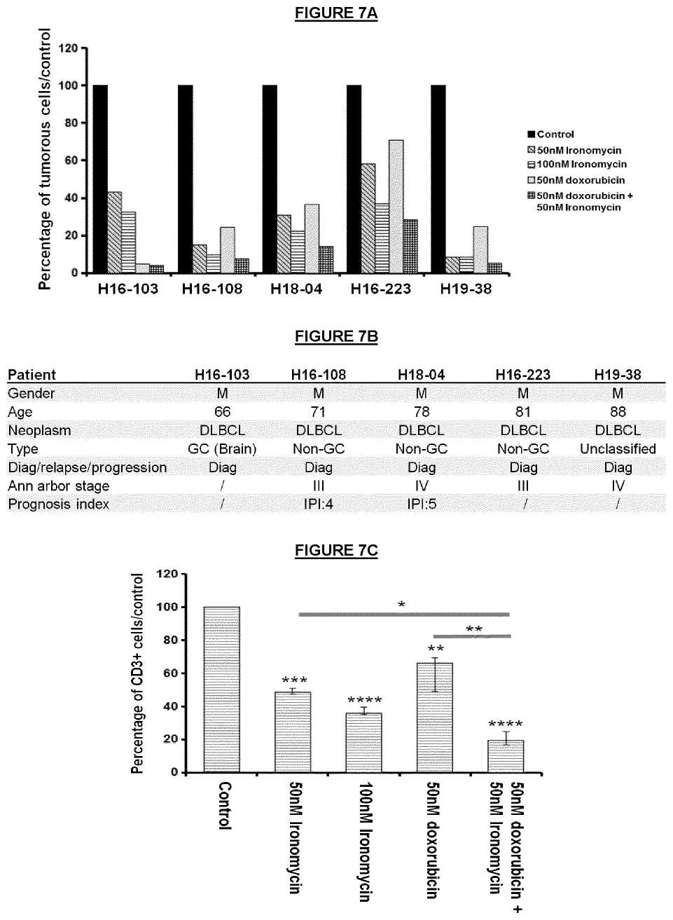

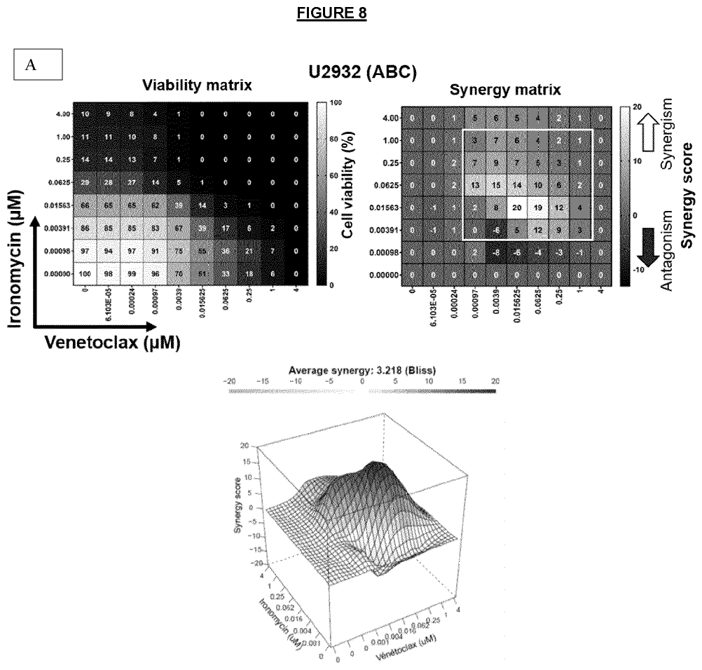

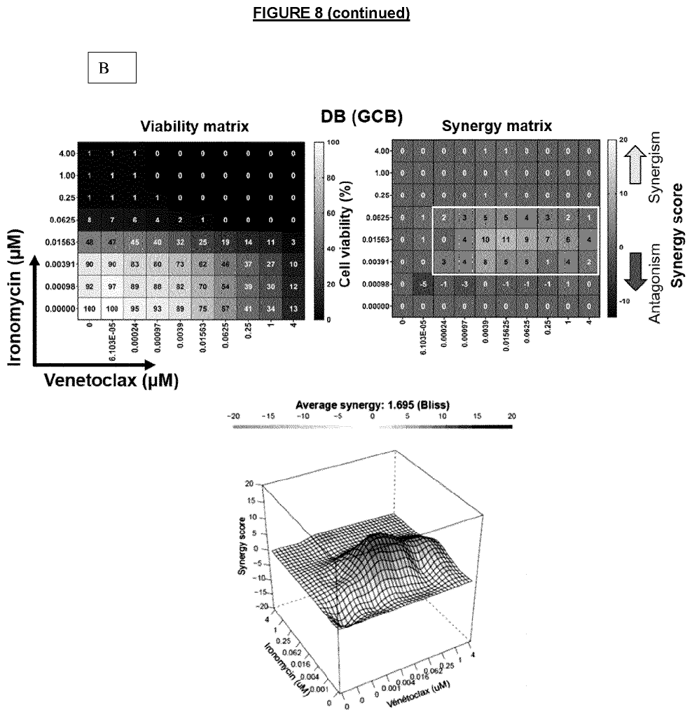

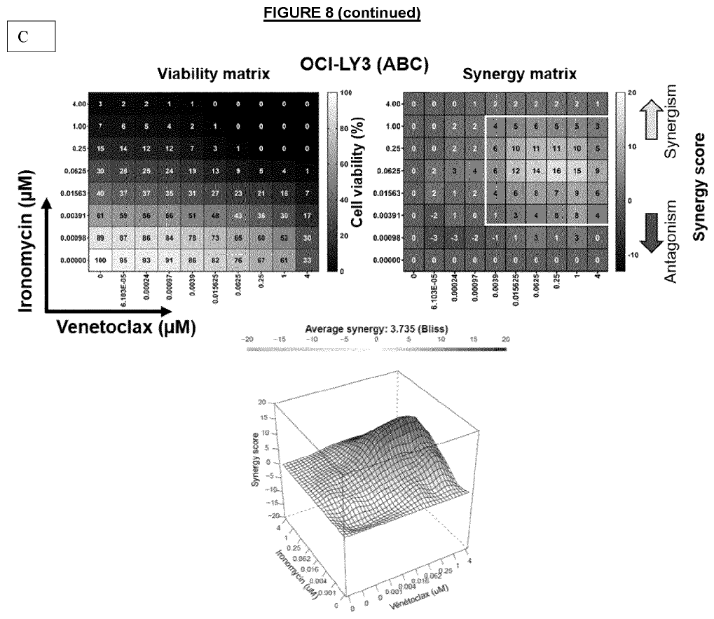

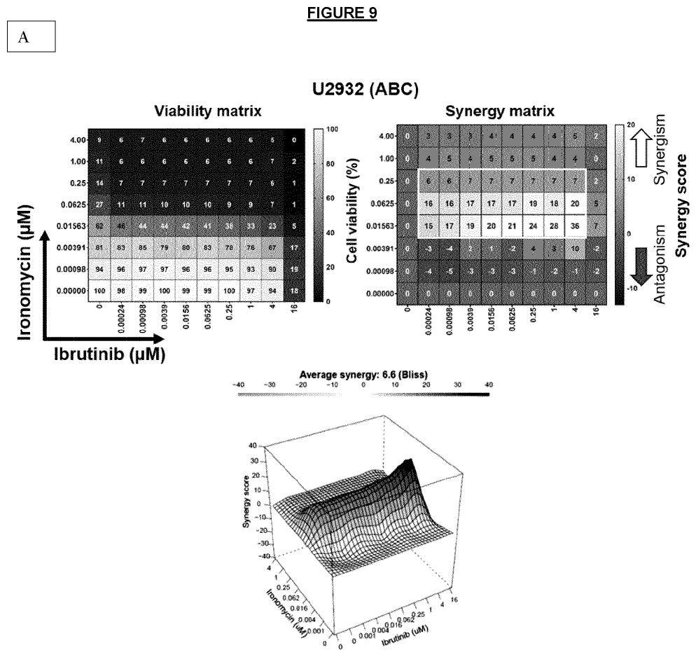

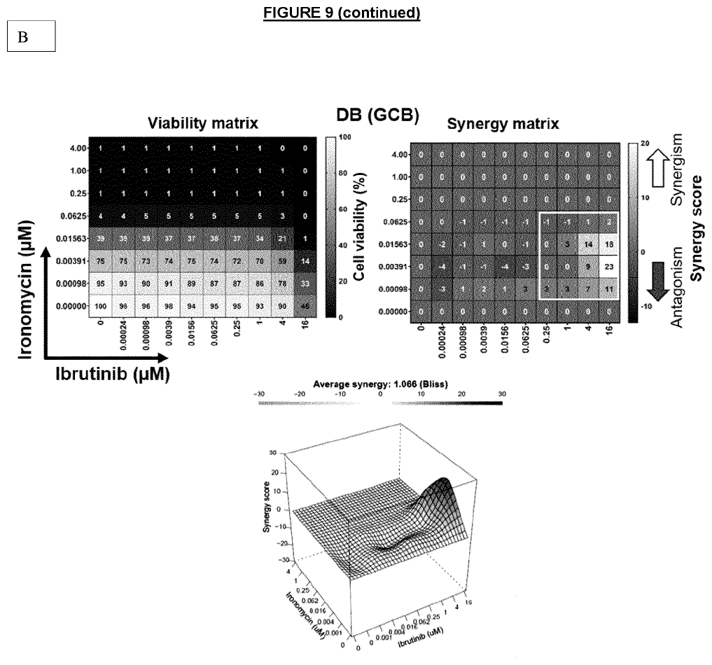

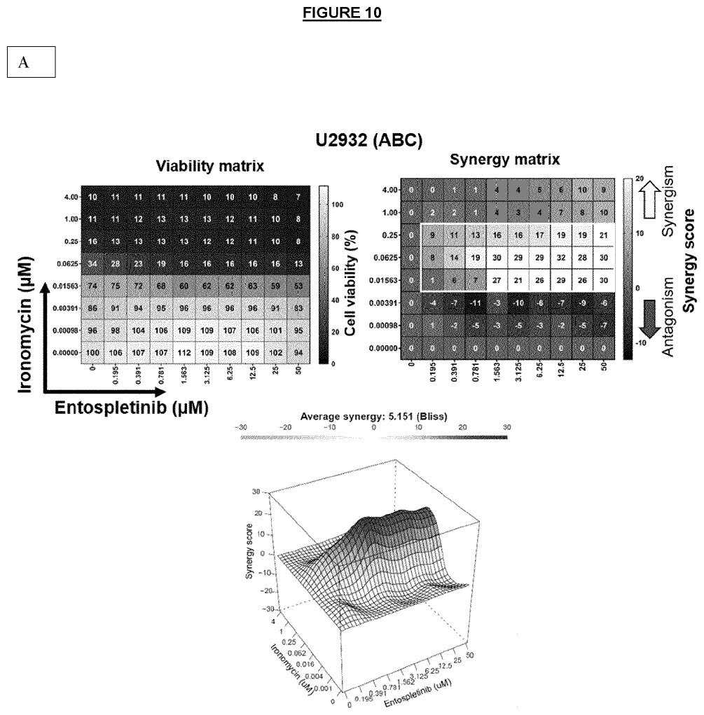

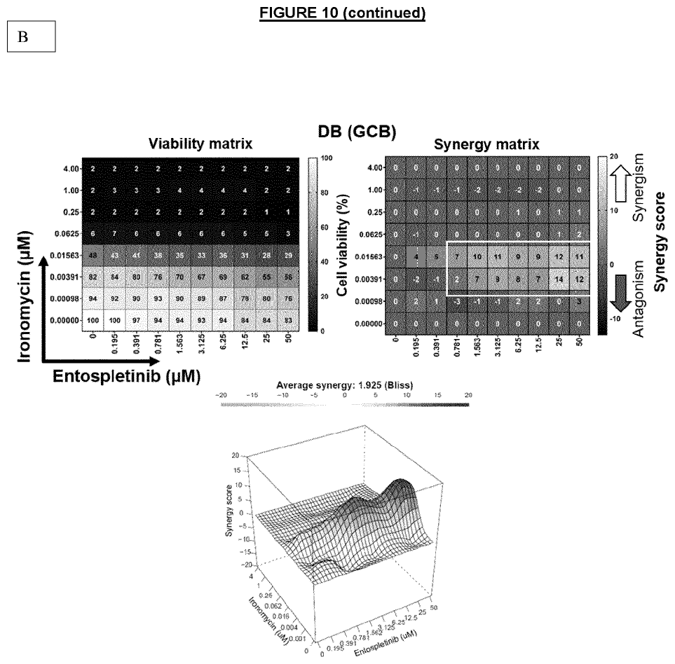

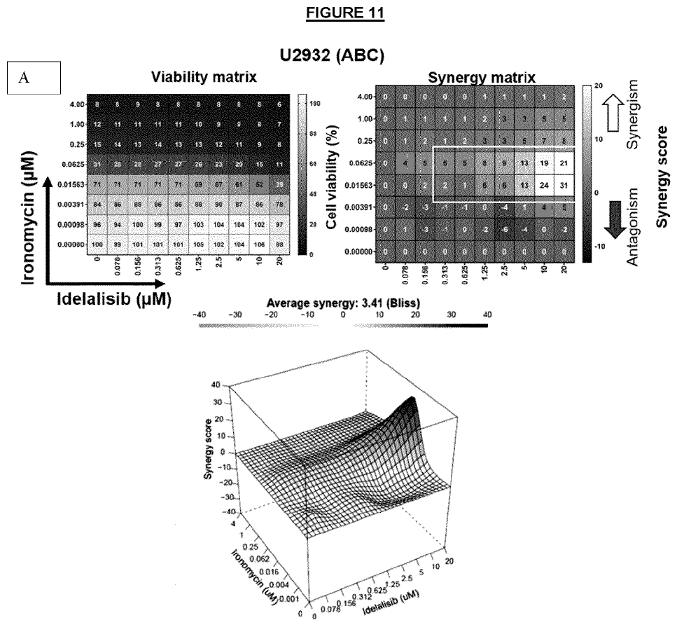

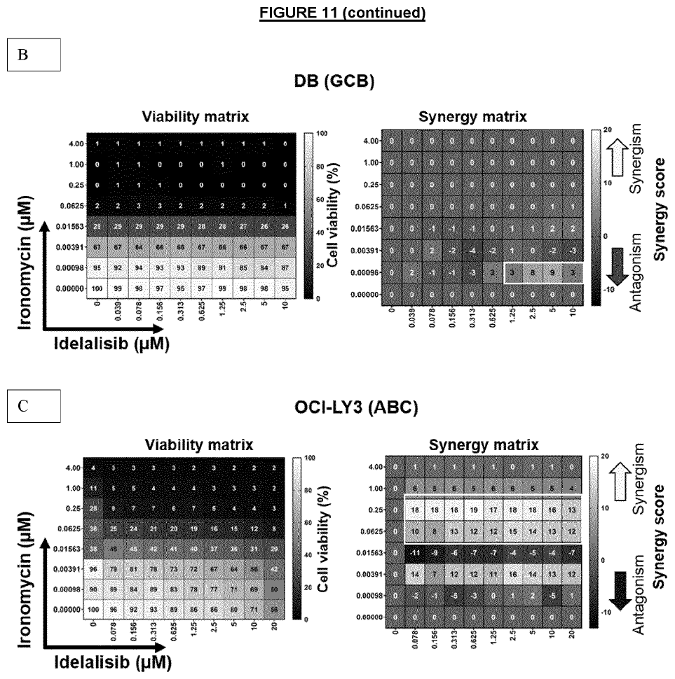

To investigate the effects of iron deprivation in DLBCL cell proliferation, OCI-LY3 and DB cell lines were treated with iron chelators and ironomycin and cell cycle distribution was assessed at 72 h. Irons chelator Deferasirox significantly affected cell cycle distribution of surviving cells with a blockade of BrdU incorporation and an increase in the G0/G1 phase (P<0.01). Same results were identified with Ironomycin (FIGS. 4A and 4B).

Given the role of iron in DNA replication, the effect of iron depletion on spontaneous DNA damage was investigated. Interestingly, iron chelators and ironomycine induced DNA double strand breaks as evidenced by γH2AX foci formation and increased phosphorylation of H2AX monitored by western blot (FIG. 4 C, D).

Eukaryotic cells contain numerous iron-requiring proteins such as iron-sulfur (Fe—S) cluster proteins, hemoproteins and ribonucleotide reductases that play key roles in DNA replication and require iron as a cofactor (Zhang, 2014). The inventors next monitored the effect of ironomycin on fork progression in DLBCL cells using DNA fiber analysis. To this end, DLBCL cell lines were pulse labeled with 10 μM IdU and 100 μM CIdU and the length of CIdU tracks was measured to estimate the distance covered by individual forks during the pulse. Interestingly, the length of CIdU tracks was significantly shorter after treatment by ironomycin compared to control cells (P<0.0001; FIGS. 5A and B). These data suggest that defective fork progression accounts for the increased spontaneous DNA damage mediated by ironomycin treatment in DLBCL cells. Gemcitabine was used as a positive control. Ironomycin iron chelation was also associated with an accumulation of DNA single-strand and replication stress, as evidenced by increased phosphorylation of replication protein A sub-unit 2 (RPA2) on threonine 21 (FIG. 4 E). Since iron was known to play a critical role in dN synthesis (; Torti and Torti, 2013), the inventors investigated the effect on dN supplementation on replication fork progression in ironomycin treated cells.

Deoxynucleotide supplementation partially reversed replication fork delay induced by Ironomycin (FIG. 5C-D).

Iron Deprivation Induces Cell Death of Primary DLBCL Cells and Potentializes with Compounds of DLBCL Treatment (Doxorubicin, Venetoclax, Ibrutinib, Entospletinib, Idelalisib).

To confirm that iron deprivation is of therapeutic interest in DLBCL, primary samples of DLBCL patients were cultured with their microenvironment recombinant CD40L in presence or absence of 50 nM and 100 nM of ironomycin. Ironomycin treatment significantly reduced the median number of viable primary DLBCL cells by 63.1% (P=0.02, N=5) and 67.1% (P=0.006, N=5) at 50 nM and 100 nM respectively (FIG. 6 B and FIGS. 7A & B). Interestingly, ironomycin demonstrated a higher toxicity in DLBCL cells compared to normal cells from the microenvironment (FIG. 7 C).

The inventors then investigated the potential of combining ironomycin with conventional drugs commonly used in DLBCL (e.g doxorubicin, etoposide, cyclophosphamide, venetoclax, ibrutinib, entospletinib, idelalisib).

Cells were treated with increasing concentration of Ironomycin and increasing concentration of conventional chemotherapy agents. Synergy matrix were built applying the Bliss equation from the viability matrix (Combes et al., 2019). Ironomycin significantly potentialized the toxicity of doxorubicin in DLBCL cells (FIG. 6 A). These data were validated with primary samples from patients (FIG. 6 B). Combination of ironomycin with doxorubicin demonstrated a significant higher toxicity compared to monotherapy (P<0.05, N=5) with a reduction of viable DLBCL cells of 92.3% (FIG. 6 B). To investigate the ironomycin-associated hematopoietic toxicity, hematopoietic progenitor colony-forming unit assays were performed with CD34+ cells from apheresis of 5 normal donors. Cells were cultured in hydroxyl-methyl-cellulose medium with or without conventional chemotherapy (5 μM of 4-OH-cyclophosphamide or 200 nM doxorubicin) or Ironomycin (10, 50 or 100 nM). Cyclophosphamide and doxorubicin induced a major toxicity of 81.8% and 99% of mean growing progenitors compared to the control. Ironomycin treatment demonstrated no significant toxicity when used at 10 nM on CFU-C, CFU-E and CFU-GM formation (N=5). Treatment with 50 and 100 nM of Ironomycin demonstrated a significant lower hematopoietic toxicity compared to conventional chemotherapy (FIG. 6 C).

The inventors tested on different DLBCL-derived cell lines (U2932, DB and OCI-LY3) the therapeutic interest to combine Ironomycin with conventional chemotherapy used in DLBCL. Interestingly, they identified a synergistic effect when Ironomycin is combined with Venetoclax Bcl2 inhibitor (FIG. 8). They also identified a synergistic effect when ironomycin is combined with Ibrutinib, a BTK inhibitor (FIG. 9), with Entospletinib, a Syk inhibitor (FIG. 10) and Idelalisib, a PI3K inhibitor (FIG. 11). These underlined a therapeutic interest to combine ironomycin with conventional drugs used in the treatment of DLBCL.

Altogether, these data demonstrate that a subgroup of high-risk DLBCL patients identified by the iron-score could benefit from targeting of iron homeostasis Ironomycin alone or in combination with doxorubicin.

### REFERENCES

Combes, E., Andrade, A. F., Tosi, D., Michaud, H. A., Coquel, F., Garambois, V., Desigaud, D., Jarlier, M., Coquelle, A., Pasero, P., et al. (2019). Inhibition of Ataxia-Telangiectasia Mutated and RAD3-related (ATR) overcomes oxaliplatin resistance and promotes anti-tumor immunity in colorectal cancer. Cancer Res.
Compagno, M., Lim, W. K., Grunn, A., Nandula, S. V., Brahmachary, M., Shen, Q., Bertoni, F., Ponzoni, M., Scandurra, M., Califano, A., et al. (2009). Mutations of multiple genes cause deregulation of NF-kappaB in diffuse large B-cell lymphoma. Nature 459, 717-721.
Ding, J., Li, T., Wang, X., Zhao, E., Choi, J. H., Yang, L., Zha, Y., Dong, Z., Huang, S., Asara, J. M., et al. (2013). The histone H3 methyltransferase G9A epigenetically activates the serine-glycine synthesis pathway to sustain cancer cell survival and proliferation. Cell Metab. 18, 896-907.
Dixon, S. J., Lemberg, K. M., Lamprecht, M. R., Skouta, R., Zaitsev, E. M., Gleason, C. E., Patel, D. N., Bauer, A. J., Cantley, A. M., Yang, W. S., et al. (2012). Ferroptosis: an iron-dependent form of nonapoptotic cell death. Cell 149, 1060-1072.
Eklund, H., Uhlin, U., FarnegArdh, M., Logan, D. T., and Nordlund, P. (2001). Structure and function of the radical enzyme ribonucleotide reductase. Prog. Biophys. Mol. Biol. 77, 177-268.
Gao, M., Monian, P., Pan, Q., Zhang, W., Xiang, J., and Jiang, X. (2016). Ferroptosis is an autophagic cell death process. Cell Res. 26, 1021-1032.
Herviou, L., Kassambara, A., Boireau, S., Robert, N., Requirand, G., Müller-Tidow, C., Vincent, L., Seckinger, A., Goldschmidt, H., Cartron, G., et al. (2018). PRC2 targeting is a therapeutic strategy for EZ score defined high-risk multiple myeloma patients and overcome resistance to IMiDs. Clin. Epigenetics 10, 121.
Isham, C. R., Tibodeau, J. D., Jin, W., Xu, R., Timm, M. M., and Bible, K. C. (2007).
Chaetocin: a promising new antimyeloma agent with in vitro and in vivo activity mediated via imposition of oxidative stress. Blood 109, 2579-2588.
Kassambara, A., Hose, D., Moreaux, J., Walker, B. A., Protopopov, A., Reme, T., Pellestor, F., Pantesco, V., Jauch, A., Morgan, G., et al. (2012). Genes with a spike expression are clustered in chromosome (sub)bands and spike (sub)bands have a powerful prognostic value in patients with multiple myeloma. Haematologica 97, 622-630.
Kassambara, A., Rbme, T., Jourdan, M., Fest, T., Hose, D., Tarte, K., and Klein, B. (2015). GenomicScape: an easy-to-use web tool for gene expression data analysis. Application to investigate the molecular events in the differentiation of B cells into plasma cells. PLoS Comput. Biol. 11, e1004077.
Lausen, B., and Schumacher, M. (1992). Maximally Selected Rank Statistics. Biometrics 48, 73-85.
Lenz, G., Wright, G., Dave, S. S., Xiao, W., Powell, J., Zhao, H., Xu, W., Tan, B., Goldschmidt, N., Iqbal, J., et al. (2008). Stromal gene signatures in large-B-cell lymphomas. N. Engl. J. Med. 359, 2313-2323.
Mai, T. T., Hamai, A., Hienzsch, A., Caneque, T., Müller, S., Wicinski, J., Cabaud, O., Leroy, C., David, A., Acevedo, V., et al. (2017). Salinomycin kills cancer stem cells by sequestering iron in lysosomes. Nat. Chem. 9, 1025-1033.
Miller, L. D., Coffman, L. G., Chou, J. W., Black, M. A., Bergh, J., D'Agostino, R., Torti, S. V., and Torti, F. M. (2011). An iron regulatory gene signature predicts outcome in breast cancer. Cancer Res. 71, 6728-6737.
Nisbet-Brown, E., Olivieri, N. F., Giardina, P. J., Grady, R. W., Neufeld, E. J., Sechaud, R., Krebs-Brown, A. J., Anderson, J. R., Alberti, D., Sizer, K. C., et al. (2003). Effectiveness and safety of ICL670 in iron-loaded patients with thalassaemia: a randomised, double-blind, placebo-controlled, dose-escalation trial. Lancet Lond. Engl. 361, 1597-1602.
Orhon, I., and Reggiori, F. (2017). Assays to Monitor Autophagy Progression in Cell Cultures. Cells 6.
Richardson, D. R., Kalinowski, D. S., Lau, S., Jansson, P. J., and Lovejoy, D. B. (2009). Cancer cell iron metabolism and the development of potent iron chelators as anti-tumour agents. Biochim. Biophys. Acta 1790, 702-717.
Rosenwald, A., Wright, G., Chan, W. C., Connors, J. M., Campo, E., Fisher, R. I., Gascoyne, R. D., Müller-Hermelink, H. K., Smeland, E. B., Giltnane, J. M., et al. (2002). The use of molecular profiling to predict survival after chemotherapy for diffuse large-B-cell lymphoma. N. Engl. J. Med. 346, 1937-1947.
Shaknovich, R., Geng, H., Johnson, N. A., Tsikitas, L., Cerchietti, L., Greally, J. M., Gascoyne, R. D., Elemento, O., and Melnick, A. (2010). DNA methylation signatures define molecular subtypes of diffuse large B-cell lymphoma. Blood 116, e81-89.
Torti, S. V., and Torti, F. M. (2013). Iron and cancer: more ore to be mined. Nat. Rev. Cancer 13, 342-355.
Yu, Y., Kovacevic, Z., and Richardson, D. R. (2007). Tuning cell cycle regulation with an iron key. Cell Cycle Georget. Tex 6, 1982-1994.
Zhang, C. (2014). Essential functions of iron-requiring proteins in DNA replication, repair and cell cycle control. Protein Cell 5, 750-760.

1.-14. (canceled)

15. An in vitro method for identifying DLBCL subject with a poor outcome that may benefit from a therapeutic treatment targeting iron metabolism, comprising the steps of:
a) measuring the expression level of at least 2 genes selected from the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject;
b) calculating a score value from said expression level obtained at step a); and
c) classifying and identifying the said subject as having a poor outcome according to the score value in comparison to a predetermined reference value (PRV).

16. The in vitro method according to claim 15, wherein the therapeutic treatment targeting iron metabolism is selected from the group consisting of iron chelators and small molecules sequestering lysosomal iron.

17. A kit dedicated to in vitro methods according to claim 15, comprising reagents for determining the expression level of at least 2 genes and/or proteins selected from the group consisting of HMOX2, PPOX, TMPRSS6, HFE, SLC25A37, STEAP1, ISCA1, HMOX1, LRP2, HIF1A, and ALAS1 in a sample of said subject.

18. The kit according to claim 17, dedicated to DLBCL subjects comprising a set of primers and/or probes for measuring the expression level of at least 5 genes and/or proteins encoded by said at least 5 genes selected from the group consisting of HMOX2, PPOX, TMPRSS6, HFE, SLC25A37, STEAP1, ISCA1, HMOX1, LRP2, HIF1A, and ALAS1.

19. A method for treating a subject having Diffuse large B-cell lymphoma (DLBCL) comprising administration of a pharmaceutical composition comprising, in a pharmaceutical acceptable vehicle, an iron chelator or a small molecule sequestering lysosomal iron.

20. The method of claim 19, wherein said subject is identified according to the following in vitro method as having a poor outcome according to iron-score and consequently likely to display a DLBCL relapse and/or death:
a) measuring the expression level of at least 2 genes selected from the group consisting of HMOX2, PPOX, TMPRSS6, HFE, SLC25A37, STEAP1, ISCA1, HMOX1, LRP2, HIF1A, and ALAS1 involved in the iron metabolism, in a biological sample obtained from said subject;
b) calculating a score value from said expression level obtained at step a); and
c) classifying and identifying the said subject as having a poor outcome according to the score value in comparison to a predetermined reference value (PRV).

21. The method of claim 19, wherein the iron chelator present in the pharmaceutical composition is a nitrogen-containing analog of salinomycin of formula (I): 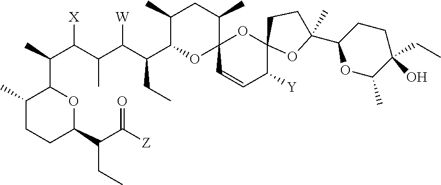
wherein:
—W is selected from the group consisting of ═O; —NR₁R₂; —NR₃—(CH₂)ₙ—NR₄R₅;
—O—(CH₂)ₙ—NR₄R₅; —NR₃—(CH₂)ₙ—N⁺R₆R₇R₈ and —O—(CH₂)ₙ—N⁺R₆R₇R₈;
—X is selected from the group consisting of ═O, —OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
—Y is selected from the group consisting of —OH; ═N—OH; —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈,
R₁ and R₂, identical or different, are selected from the group consisting of H; (C₁-C₁₆)-alkyl; (C₃-C₁₆)-alkenyl; (C₃-C₁₆)-alkynyl; (C₃-C₁₆)-cycloalkyl; aryl; heteroaryl; (C₁-C₆)-alkyl-aryl; (C₁-C₆)-alkyl-heteroaryl; or R₁ represents H and R₂ represents OR₉, where R₉ is H, (C₁-C₆)-alkyl, aryl and (C₁-C₆)-alkyl-aryl;
R₃ is selected from the group consisting of H; (C₁-C₆)-alkyl; (C₁-C₆)-alkyl-aryl;
R₄ and R₅, identical or different, are selected from the group consisting of H; (C₁-C₆)-alkyl; aryl and (C₁-C₆)-alkyl-aryl;
R₆, R₇ and R₈, identical or different, are selected from the group consisting of (C₁-C₆)-alkyl; aryl and (C₁-C₆)-alkyl-aryl;
—Z is a group such as OH; NHNR₉R₁₀; NHOC(O)R₁₁; N(OH)—C(O)R₁₁; OOH,
SR₁₂; 2-aminopyridine; 3-aminopyridine; —NR₃—(CH₂)ₙ—NR₄R₅; and —NR₃—(CH₂)ₙ—OH; where:
R₉ and R₁₀, identical or different, are selected from the group consisting of H, (C₁-C₆)-alkyl, aryl and (C₁-C₆)-alkyl-aryl;
R₁₁ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl;
R₁₂ is selected from the group consisting of H; (C₁-C₁₆)-alkyl; (C₃-C₁₆)-alkenyl; (C₃-C₁₆)-alkynyl; aryl; heteroaryl; (C₁-C₆)-alkyl-aryl; (C₁-C₆)-alkyl-heteroaryl
n=0, 2, 3, 4, 5 or 6,
with the proviso that at least one of W, X and Y is selected from the group consisting of —NR₁R₂; —NR₃—(CH₂)ₙ—NR₄R₅; —O—(CH₂)ₙ—NR₄R₅; —NR₃—(CH₂)ₙ—N⁺R₆R₇R₈ and —O—(CH₂)ₙ—N⁺R₆R₇R₈.

22. The method of claim 21, wherein the iron chelator present in the pharmaceutical composition is a nitrogen-containing analog of salinomycin of formula (I): 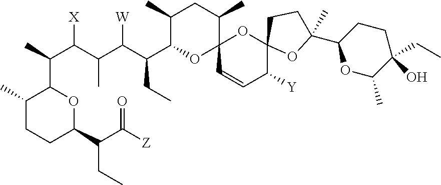
wherein X is OH, Z is OH, and Y is NR₁R₂ where R₁ is H and R₂ is selected from the group consisting of (C₁-C₁₆)-alkyl, (C₃-C₁₆)-alkenyl, (C₃-C₁₆)-alkynyl, and (C₃-C₁₆)-cycloalkyl.

23. The method of claim 22, wherein the iron chelator present in the pharmaceutical composition is a nitrogen-containing analog of salinomycin of formula (I): 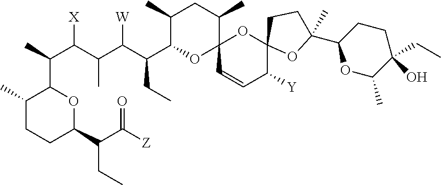
wherein W is =O, X is OH, Z is OH, and Y is NR₁R₂ where R₁ is H and R₂ is selected from the group consisting of (C₃-C₅)-alkynyl and (C₃-C₆)-cycloalkyl.

24. The method of claim 19, wherein said composition further comprises at least one other anti-cancer agent selected from the group consisting of agents used in chemotherapy, targeted treatments, immune therapies, and combinations thereof, wherein administration of (i) said iron chelator or small molecule sequestering lysosomal iron, and (ii) said anti-cancer agent is simultaneous, separate, or staggered.

25. The method of claim 24, wherein the iron chelator or small molecule sequestering lysosomal iron is selected in the group consisting of Deferasirox, Deferoxamine, Deferiprone, Salinomycin, analogs or derivatives thereof; and the other anti-cancer agent is selected from the group consisting of agents used in chemotherapy.

26. The method of claim 25, wherein the iron chelator is a nitrogen-containing analog of salinomycin of formula (I) 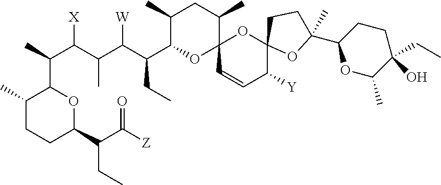
wherein W is =O, X is OH, Z is OH, and Y is NR₁R₂ where R₁ is H and R₂ is selected from the group consisting of (C₃-C₅)-alkynyl and (C₃-C₆)-cycloalkyl, and the other chemotherapy compound is Doxorubicin, Venetoclax, Idelalisib, Ibrutinib, or entospletinib.

27. A pharmaceutical product or composition comprising:
(i) a nitrogen-containing analog of salinomycin of formula (I) 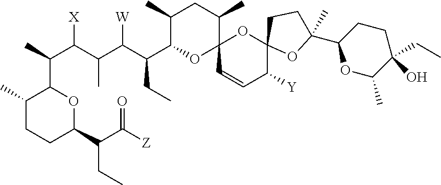
wherein W is =O, X is OH, Z is OH, and Y is NR₁R₂ where R₁ is H and R₂ is selected from the group consisting of (C₃-C₅)-alkynyl and (C₃-C₆)-cycloalkyl, and
(ii) a chemotherapy compound selected from the group consisting of cyclophosphamide, doxorubicin, etoposide, Idelalisib, Ibrutinib, and entospletinib.
